(19) United States
(12) Patent Application Publication
(10) Pub. No.: US 2021/0162070 A1
(43) Pub. Date: Jun. 3, 2021

(54) Gene Therapy for Muscle Improvement

(71) Applicant: The Board of Trustees of the Leland Stanford Junior University, Stanford, CA (US)

(72) Inventor: Michele P. Calos, Menlo Park, CA (US)

(21) Appl. No.: 16/649,883

(22) PCT Filed: Oct. 29, 2018
(86) PCT No.: PCT/US2018/058034

§ 371 Date: Mar. 23, 2020

Related U.S. Application Data
(60) Provisional application No. 62/578,973, filed on Oct. 30, 2017.

(51) Int. Cl. A61K 48/00; A61K 38/46; C12N 15/85

(52) U.S. Cl. CPC A61K 48/0016; A61K 48/0075; C12N 15/85; A61K 38/465; A61K 48/0083

(57) ABSTRACT

The present disclosure provides methods for altering a phenotypic characteristic of muscular dystrophy, treating muscular dystrophy, and/or alleviating a symptom of muscular dystrophy. Methods for integrating a polynucleotide sequence into the genome of a human cell are provided. The present methods result in alteration of the phenotypic characteristic of muscular dystrophy, treatment of muscular dystrophy, and/or alleviating a symptom of muscular dystrophy. Also provided are nucleic acids that include sequences for integrating a polynucleotide sequence of interest into the genome of a human cell. A transgenic human cell including site specific recombination sites is also disclosed.

### CROSS-REFERENCE

This application claims the benefit of U.S. Provisional Patent Application No. 62/578,973 filed Oct. 30, 2017, which application is incorporated herein by reference in its entirety.

### INTRODUCTION

Muscular dystrophies are genetic diseases caused by mutations in genes encoding muscle proteins, leading to progressive muscle degeneration. Most of the disease genes are recessive, making these disorders candidates for gene therapy. It is desirable to effectively treat a subject with a muscular disorder like muscular dystrophy. In many instances, it is also desirable to effectively insert a nucleic acid into the human genome such that the inserted nucleic acid may be expressed while causing no disruption of the regulatory elements or genes in the genome at or near the insertion site.

However, the currently available technologies for treating muscular dystrophy have serious drawbacks. For example, gene therapy using a virus-mediated delivery system in a subject involves a viral capsid of fixed dimensions and foreign proteins, which create limits with respect to the size of nucleic acid that can be delivered and a subject's potential immune response to the foreign proteins. Treatment mediated by retroviruses, lentiviruses, transposons, and non-homologous end-joining also results in random integration. The consequent lack of control over transgene insertion site, copy number, and orientation compromises the precision of experiments. New methods for treating muscular disorders in a site specific manner are needed to address serious drawbacks.

The methods, nucleic acids, and transgenic human cells disclosed herein address the above limitations and fulfill other needs.

### SUMMARY

The present disclosure provides methods for altering a phenotypic characteristic of muscular dystrophy, of treating muscular dystrophy, and/or alleviating a symptom of muscular dystrophy. Methods for integrating a polynucleotide sequence into the genome of a human cell are provided. The present methods result in alteration of the phenotypic characteristic of muscular dystrophy, treatment of muscular dystrophy, and/or alleviating a symptom of muscular dystrophy. Also provided are nucleic acids that include sequences for integrating a polynucleotide sequence of interest into the genome of a human cell. A transgenic human cell including site specific recombination sites is also disclosed.

In certain embodiments, a method of altering a phenotypic characteristic of muscular dystrophy, includes: introducing into the human cell a circular nucleic acid comprising one or more polynucleotide sequences, wherein the one or more polynucleotide sequences comprise at least a follistatin (FST) gene sequence and one of a calpain 3 (CAPN3) gene sequence, an alpha-sarcoglycan (SGCA) gene sequence, or a dysferlin (DYSF) gene sequence; and expressing the one or more polynucleotide sequences in the circular nucleic acid in the human cell, wherein the expressing results in altering the phenotypic characteristic of muscular dystrophy. In certain embodiments, the methods described herein are nonviral.

In other embodiments, a method of treating muscular dystrophy, includes: introducing into the human cell a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence; and expressing the one or more polynucleotide sequences in the circular nucleic acid in the human cell, wherein the expressing results in treating muscular dystrophy.

In certain embodiments, a method of alleviating a symptom of muscular dystrophy, includes: introducing into the human cell a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence; and expressing the one or more polynucleotide sequences in the circular nucleic acid in the human cell, wherein the expressing results in alleviating the symptom of muscular dystrophy.

In certain embodiments, the introducing into the human cell provides for integration of the circular nucleic acid into a genome of the human cell. In certain embodiments, the integration of the circular nucleic acid into the genome of the human cell involves an integrase. In certain embodiments, the integrase is a polypeptide. In certain embodiments, the integrase is a phiC31 integrase. In other embodiments, the integration involves a genome-editing enzyme. In certain embodiments, the genome-editing enzyme is a polypeptide. In certain embodiments, the genome-editing enzyme is Cas9 polypeptide, a zinc finger nuclease, a TALEN, or an enzymatically inactive type II CRISPR/Cas polypeptide. In other embodiments, the integration involves a RNA-guided endonuclease. In certain embodiments, the RNA-guided endonuclease is a polypeptide. In certain embodiments, the RNA-guided endonuclease is selected from a type II CRISPR/Cas polypeptide, a type V CRISPR/Cas polypeptide, and a type VI CRISPR/Cas polypeptide.

In certain embodiments, a method of delivering one or more polynucleotides into a human cell of a target tissue includes: creating an opening in the subject; introducing the circular nucleic acid including the one or more polynucleotide sequences into the target tissue; and applying electroporation to the target tissue. In certain embodiments, a method of delivering one or more polynucleotides into a human cell of a target tissue includes: positioning a device in contact with a subject to restrict blood flow to a limb; creating an opening in the subject to expose a vessel; introducing a circular nucleic acid including the one or more polynucleotides into the vessel; releasing the device; and closing the opening. Examples of methods of delivering a polynucleotide into a human cell of a target tissue may be found in, for example, Portlock et al. (2006), Bertoni et al. (2006), Wolf et al. (1990), and Hagstrom et al. (2004), the disclosures of which are incorporated herein by reference. In certain embodiments, the circular nucleic acid includes a promoter and a reporter gene.

In certain embodiments, a method of inserting a polynucleotide sequence into a genome of a human cell further includes inserting one or more polynucleotide sequences into a genome of a human cell, comprising: introducing into the human cell: a circular nucleic acid with one or more polynucleotide sequences flanked by a first recombination site of a first integrase and a first recombination site of a second integrase; the first integrase; and the second integrase, wherein the human cell includes a second recombination site of the first integrase and a second recombination site of the second integrase at a locus in a chromosome; maintaining the human cell under conditions that facilitate recombination between first recombination site of the first integrase and the second recombination site of the first integrase and between the first recombination site of the second integrase and the second recombination site of the second integrase, wherein the introducing and maintaining results in insertion of the one or more polynucleotide sequences into the genome of the human cell at the locus. Examples of methods of inserting a polynucleotide sequence into a genome of a human cell may be found in, for example, in PCT publication WO2015073703, the disclosure of which is incorporated herein by reference.

In certain embodiments, the first and second integrases may be introduced into the human cell by introducing a nucleic acid encoding the first and second integrases into the cell. In certain embodiments, the first and second integrases are introduced into the human cell by introducing a first nucleic acid encoding the first integrase and a second nucleic acid encoding the second integrase. The nucleic acid encoding the first and/or the second integrases may be an mRNA or a circular DNA. In certain embodiments, the first integrase is phiC31. In certain embodiments, the second integrase is Bxb1. In certain embodiments, the phiC31 recombination site may be attB and the phiC31 second recombination site may be attP. In other embodiments, the phiC31 first recombination site may be attP and the phiC31 second recombination site may be attB. In certain embodiments, the Bxb1 first recombination site may be attB and the Bxb1 second recombination site may be attP. In other embodiments, the Bxb1 first recombination site may be attP and the Bxb1 second recombination site may be attB.

Exemplary human cells include muscle cells, neuronal cells, and pluripotent stem (PS) cells, such as, embryonic stem (ES) cells and induced pluripotent stem (iPS) cells.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present invention.

FIGS. 1A-1D illustrate plasmid and DNA vascular injection, including plasmid constructs and live imaging.

FIGS. 2A-2E depict a comparison of single and double vascular injections of a subject nucleic acid in mice in a 4-week experiment.

FIGS. 3A-3E depict a comparison of single and double vascular injections of a subject nucleic acid in mice in a 12-week experiment.

FIGS. 4A-4D show further analysis of a 12-week gene therapy experiment in mice including immunohistochemistry data.

FIG. 5 depicts a calpain 3 (CAPN3) expression plasmid used according to the methods described herein.

FIG. 6 depicts a follistatin expression plasmid used according to the methods described herein.

FIG. 7 depicts luciferase live images of mice indicating the efficiency of gene delivery of the CAPN3 expression plasmid for calpain 3.

FIG. 8 depicts Western blot results performed to detect CAPN3 expression.

FIG. 9 depicts results of an ELISA assay to verify production of follistatin.

FIG. 10 depicts reducing the increase of centronucleated fibers as a result of gene therapy with the CAPN3 and follistatin expression plasmids.

### DEFINITIONS

As used herein, “altering” can mean being modified by at least 10%, 20%, 30%, 40%, 50%, 60%, 70%, 80%, 90%, or 100% relative to an initial value. In certain embodiments of the invention the phenotype is modified by at least 2, 3, 5, 10, 20, 50, or 100-fold relative to an initial value.

As used herein, a “phenotypic characteristic” can be any observable or detectable characteristic, property, attribute, or function of a cell. The phenotypic characteristic may be observed or detected in any of a number of ways. For example, a phenotypic characteristic may be observed or detected either by performing a test, observation, or measurement on the cell itself or by performing a test, observation, or measurement, on other cells, tissues, organs, etc., that may be affected by the cell, or by performing a test, observation, or measurement on a subject that contains the cell. The term “phenotypic characteristic” includes any “phenotypic characteristic” and also refers more broadly to characteristics, properties, attributes, functions, etc., that may result from a combination of two or more phenotypic characteristics. Certain of these phenotypic characteristics may be defined with respect to an effect that human cells exhibiting the phenotypic characteristic have on other cells or tissues either in vitro or in vivo.

“Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease.

As used herein, “alleviating” can mean being reduced by at least 10%, 20%, 30%, 40%, 50%, 60%, 70%, 80%, 90%, or 100% relative to an initial value. In certain embodiments of the invention the symptom is modified by at least 2, 3, 5, 10, 20, 50, or 100-fold relative to an initial value.

By “nucleic acid construct” it is meant a nucleic acid sequence that has been constructed to comprise one or more functional units not found together in nature. Examples include circular, double-stranded, extrachromosomal DNA molecules (plasmids), cosmids (plasmids containing COS sequences from lambda phage), viral genomes comprising non-native nucleic acid sequences, and the like.

As used herein, “nucleic acid fragment of interest” or “polynucleotide sequence of interest” refers to any nucleic acid fragment that one wishes to insert into a genome. Examples of nucleic acid fragments of interest include any genes (e.g., RNA encoding, protein-encoding), such as therapeutic genes, marker genes, control regions, trait-producing fragments, and the like.

A “coding sequence” or a sequence which “encodes” a selected polypeptide, is a nucleic acid molecule which is transcribed (in the case of DNA) and translated (in the case of mRNA) into a polypeptide, for example, in vivo when placed under the control of appropriate regulatory sequences (or “control elements”). The boundaries of the coding sequence are typically determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence. Other “control elements” such a regulatory sequences, e.g., promoter sequences may also be associated with a coding sequence.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

“Homozygous” state means a genetic condition existing when identical alleles reside at corresponding loci on homologous chromosomes. In contrast, “heterozygous” state means a genetic condition existing when different alleles reside at corresponding loci on homologous chromosomes.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

An “expression cassette” comprises any nucleic acid construct capable of directing the expression of a gene/coding sequence of interest. Such cassettes can be constructed into a “vector,” “vector construct,” “expression vector,” or “gene transfer vector,” in order to transfer the expression cassette into target cells. Thus, the term includes cloning and expression vehicles, as well as viral vectors.

A cell has been “transformed” or “transfected” by exogenous or heterologous DNA, e.g. a DNA construct, when such DNA has been introduced inside the cell. The transforming DNA may or may not be integrated (covalently linked) into the genome of the cell.

“Locus” refers to a specific location on a chromosome. A known locus can contain known genetic information, such as one or more polymorphic marker sites.

By “recombination” it is meant a process of exchange of genetic information between two polynucleotides. As used herein, “homologous recombination (HR)” refers to the specialized form of such exchange that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homologous recombination may result in an alteration of the sequence of the target molecule, if the donor polynucleotide differs from the target molecule and part or all of the sequence of the donor polynucleotide is incorporated into the target polynucleotide.

By “integration” it is meant that the gene of interest is stably inserted into the cellular genome, i.e., covalently linked to the nucleic acid sequence within the cell's chromosomal DNA. By “targeted integration” it is meant that the gene of interest is inserted into the cell's chromosomal or mitochondrial DNA at a specific site, or “integration site”.

A “DNA molecule” refers to the polymeric form of deoxyribonucleotides (adenine, guanine, thymine, or cytosine) in either single stranded form or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

As used herein, a “target locus” is a region of DNA into which a gene or polynucleotide of interest is integrated, e.g., a region of chromosomal or mitochondrial DNA in a cell.

As used herein, the term “reporter gene” refers to a coding sequence whose product may be assayed easily and quantifiably when attached to promoter and in some instances enhancer elements and introduced into tissues or cells. The promoter may be a constitutively active promoter or it may be an inducible promoter.

By “targeted nuclease” it is meant a nuclease that is targeted to a specific DNA sequence. Targeted nucleases are targeted to a specific DNA sequence by the DNA binding domain to which they are fused. In other words, the nuclease is guided to a DNA sequence, e.g. a chromosomal sequence or an extrachromosomal sequence, e.g. an episomal sequence, a minicircle sequence, a mitochondrial sequence, a chloroplast sequence, etc., by virtue of its fusion to a DNA binding domain with specificity for the target DNA sequence of the target locus of interest.

By “pluripotent stem cell” or “pluripotent cell” it is meant a cell that has the ability under appropriate conditions of producing progeny of several different cell types that are derivatives of all of the three germinal layers (endoderm, mesoderm, and ectoderm). Pluripotent stem cells are capable of forming teratomas. Examples of pluripotent stem cells are embryonic stem (ES) cells, embryonic germ stem (EG) cells, and induced pluripotent stem (iPS) cells. PS cells may be from any organism of interest, including, e.g., human.

By “embryonic stem cell” or “ES cell” it is meant a cell that a) can self-renew, b) can differentiate to produce all types of cells in an organism, and c) is derived from a developing organism or is an established ES cell line which was derived from a developing organism. ES cell may be derived from the inner cell mass of the blastula of a developing organism. ES cell may be derived from a blastomere generated by single blastomere biopsy (SBB) involving removal of a single blastomere from the eight cell stage of a developing organism. In general, SBB provides a non-destructive alternative to inner cell mass isolation. SBB and generation of hES cells from the biopsied blastomere is described in Cell Stem Cell, 2008 Feb. 7; 2(2):113-7. ES cells can be cultured over a long period of time while maintaining the ability to differentiate into all types of cells in an organism. In culture, ES cells typically grow as flat colonies with large nucleo-cytoplasmic ratios, defined borders and prominent nuclei. In addition, ES cells express SSEA-3, SSEA-4, TRA-1-60, TRA-1-81, and Alkaline Phosphatase, but not SSEA-1. Examples of methods of generating and characterizing ES cells may be found in, for example, U.S. Pat. Nos. 7,029,913, 5,843,780, and 6,200,806, the disclosures of which are incorporated herein by reference.

By “embryonic germ stem cell”, embryonic germ cell” or “EG cell” it is meant a cell that a) can self-renew, b) can differentiate to produce all types of cells in an organism, and c) is derived from germ cells and germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, the disclosures of which are incorporated herein by reference.

By “induced pluripotent stem cell” or “iPS cell” it is meant a cell that a) can self-renew, b) can differentiate to produce all types of cells in an organism, and c) is derived from a somatic cell. iPS cells have an ES cell-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. iPS cells may be generated by providing the cell with “reprogramming factors”, i.e., one or more, e.g., a cocktail, of biologically active factors that act on a cell to alter transcription, thereby reprogramming a cell to pluripotency. Examples of methods of generating and characterizing iPS cells may be found in, for example, Application Nos. US20090047263, US20090068742, US20090191159, US20090227032, US20090246875, and US20090304646, the disclosures of which are incorporated herein by reference.

By “somatic cell” it is meant any cell in an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include both neurons and neural progenitors, the latter of which may be able to self-renew and naturally give rise to all or some cell types of the central nervous system but cannot give rise to cells of the mesoderm or endoderm lineages.

As used herein, the terms “enriched” and “enrichment” may be used interchangeably to refer to the process of increasing the ratio of target entities (e.g., genetically modified cells including a target polynucleotide sequence) to non-target entities (e.g., cells not including the target polynucleotide sequence) in a genetically modified sample compared to the ratio in the original sample.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a human cell” includes a plurality of such human cells and reference to “the polynucleotide sequence of interest” includes reference to one or more polynucleotide sequences of interest and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for altering a phenotypic characteristic of muscular dystrophy, treating muscular dystrophy, and/or alleviating a symptom of muscular dystrophy. Methods for integrating a polynucleotide sequence into the genome of a human cell are provided. The present methods result in alteration of the phenotypic characteristic of muscular dystrophy, treatment of muscular dystrophy, and/or alleviating a symptom of muscular dystrophy. Also provided are nucleic acids that include sequences for integrating a polynucleotide sequence of interest into the genome of a human cell. A transgenic human cell including site specific recombination sites is also disclosed.

Method of Altering a Phenotypic Characteristic of Muscular Dystrophy, Treating Muscular Dystrophy, and/or Alleviating a Symptom of Muscular Dystrophy

In certain embodiments, a method of altering a phenotypic characteristic of muscular dystrophy, includes: introducing into the human cell a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence; and expressing the one or more polynucleotide sequences in the circular nucleic acid in the human cell, wherein the expressing results in altering the phenotypic characteristic of muscular dystrophy.

Non-limiting examples of phenotypic characteristics that can be modulated (e.g., increased, decreased, temporally or spatially altered) by the present invention are: (i) expression of one or more genes; (ii) secretion of one or more molecules; (iii) migration to one or more sites in the body; (iv) ability to cause an alteration in one or more phenotypic characteristics or phenotypic characteristics of another human cell (e.g. muscle cell) or ability to cause an alteration in an alteration in one or more phenotypic characteristics or phenotypic characteristics of a non-muscle-related cell, etc. A non-exhaustive list of the phenotypic characteristics that can be modified by an effector include pro-inflammatory, anti-inflammatory, immunogenic, tolerogenic, tissue destructive, tissue restorative, cytotoxic, migratory, bone-resorbing, pro-angiogenic, anti-angiogenic, suppressor, antigen presenting, or phagocytic. Additionally, many changes brought about by effector molecules cannot easily be assigned to one of these phenotypic characteristics but are still therapeutically relevant. Methods for observing, detecting, measuring, etc., these phenotypic characteristics and phenotypic characteristics are known in the art. For example, gene expression profiles can be assessed at the RNA level using cDNA or oligonucleotide microarray analysis, Northern blots, RT-PCR, etc. Protein expression can be measured using, for example, immunoblotting, immunohistochemistry, protein microarrays, etc. Various cell-based assays and animal models may be used.

In other embodiments, a method of treating muscular dystrophy, includes: introducing into the human cell a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence; and expressing the one or more polynucleotide sequences in the circular nucleic acid in the human cell, wherein the expressing results in treating muscular dystrophy.

In certain embodiments, a method of alleviating a symptom of muscular dystrophy, includes: introducing into the human cell a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence; and expressing the one or more polynucleotide sequences in the circular nucleic acid in the human cell, wherein the expressing results in alleviating the symptom of muscular dystrophy.

Symptoms of muscular dystrophy which may be used to determine whether a subject has muscular dystrophy include progressive muscle wasting (loss of muscle mass), poor balance, frequent falls, walking difficulty, waddling gait, calf pain, limited range movement, muscle contractures, respiratory difficulty, drooping eyelids (ptosis), gonadal atrophy and scoliosis (curvature of the spine). Other symptoms can include centronucleation, cardiomyopathy, and arrhythmias. Symptoms of myotonic dystrophy which may be used to determine whether a subject has myotonic dystrophy include abnormal stiffness of muscles and myotonia (difficulty or inability to relax muscles). Other symptoms of myotonic dystrophy include weakening and wasting of muscles (where the muscles shrink over time), cataracts, and heart problems. Myotonic dystrophy affects heart muscle, causing irregularities in the heartbeat. It also affects the muscles of the digestive system, causing constipation and other digestive problems. Myotonic dystrophy may cause cataracts, retinal degeneration, low IQ, frontal balding, skin disorders, atrophy of the testicles, insulin resistance and sleep apnea. A muscle disease of muscular dystrophy may be diagnosed on the basis of symptoms and characteristic traits such as those described above and/or on the results of a muscle biopsy, DNA or blood test. Blood tests work by determining the level of creatine phosphokinase (CPK). Other tests may include serum CPK, electromyography and electrocardiography. Muscular dystrophies can also alter the levels of myoglobin, LDH, creatine, AST and aldolase.

In certain embodiments, the introducing into the human cell provides for integration of the circular nucleic acid into a genome of the human cell. In certain embodiments, the integration of the circular nucleic acid into the genome of the human cell involves an integrase. In certain embodiments, the integrase is a polypeptide. In certain embodiments, the integrase is a phiC31 integrase. In other embodiments, the integration involves a genome-editing enzyme. In certain embodiments, the genome-editing enzyme is a polypeptide. In other embodiments, the integration involves a RNA-guided endonuclease. In certain embodiments, the RNA-guided endonuclease is a polypeptide. In certain embodiments, the circular nucleic acid comprises a promoter and a reporter gene.

The present disclosure also provides methods of treating muscular dystrophy in a subject. Such methods generally involve delivering to a subject in need with a circular nucleic acid as disclosed above. Subjects suitable for therapy include any subject having or at risk of muscular dystrophy. Subjects include mammals, including both human and non-human mammals, e.g., primates, rodents, cows, horses, pigs, sheep, etc.

### Polynucleotide Sequences

In certain embodiments, the one or more polynucleotide sequences include an FST gene. FST causes general muscle growth and reduces fibrosis; however FST alone as a single therapy may be insufficient for maximal therapeutic benefit. In certain embodiments, the one or more polypeptides include a CAPN3 gene, alone, or in combination with, an FST gene. In certain embodiments, the one or more polypeptides include an SGCA gene, alone, or in combination with, an FST gene. In certain embodiments, the one or more polypeptides include an MSX1 gene, alone, or in combination with, an FST gene. The sequence encoding for FST, CAPN3, SGCA, and/or MSX1 are inserted in a plasmid. In certain embodiments, the plasmid is therapeutic. In certain embodiments, the plasmid further includes an expression cassette, a resistance gene, and/or a detectable signal.

In certain embodiments, the one or more polypeptides delivered into a human cell of a target tissue include a DYSF gene, alone, or in combination with, an FST gene. DYSF is involved in the repair of the sarcolemmal membrane and loss of DYSF leads to fragile sarcolemmal membranes that can be detected by permeability to certain dye including, but not limited to, Evan's blue dye.

In certain embodiments, the polynucleotide sequence encoding for FST may be used alone, or in combination with, other polypeptides, including but not limited to, CAPN3, SGCA, and/or DYSF. The nucleic acid may be, for example, a circular nucleic acid inside of a cell in vitro, a circular nucleic acid inside of a cell ex vivo, or a circular nucleic acid inside of a cell in vivo.

A nucleic acid composition may encode for FST alone, or encode for one or more other polypeptides, including CAPN3, SGCA, and/or DYSF. In certain cases, the coding sequence for CAPN3, SGCA, and/or DYSF replace an element of the nucleic acid composition. In certain cases, the element replaced is a resistance gene. In certain cases, the coding sequence for CAPN3, SGCA, and/or DYSF does not replace an element of the nucleic acid composition. In some cases, a nucleic acid composition includes any combination of polynucleotide sequences, alone, or in combination with each other.

In other embodiments, the one or more polypeptides include, but not limited to, those derived from the actin and myosin gene families, such as from the MyoD gene family, DYSF, MSX1, CAPN3, SGCA, the myocyte-specific enhancer binding factor MEF-2, control elements derived from the human skeletal actin gene, the cardiac actin gene, muscle creatine kinase sequence elements and the murine creatine kinase enhancer (mCK) element, control elements derived from the skeletal fast-twitch troponin C gene, the slow-twitch cardiac troponin C gene and the slow-twitch troponin I gene: hypozia-inducible nuclear factors, steroid-inducible elements and promoters including the glucocorticoid response element (GRE), and other polynucleotides.

Vectors may be provided directly to the subject cells. In other words, the cells are contacted with vectors comprising the polynucleotide sequences encoding one or more polypeptides such that the vectors are taken up by the cells. Vectors used for providing the nucleic acids encoding one or more polypeptides to the subject cells will typically comprise suitable promoters for driving the expression, that is, transcriptional activation, of the nucleic acid of interest. In other words, the nucleic acid of interest will be operably linked to a promoter. This may include ubiquitously acting promoters, for example, the CAG promoter, or inducible promoters, such as promoters that are active in particular cell populations or that respond to the presence of drugs such as tetracycline. By transcriptional activation, it is intended that transcription will be increased above basal levels in the target cell by 10 fold, by 100 fold, more usually by 1000 fold. In addition, vectors used for providing one or more polynucleotide sequences to the subject cells may include nucleic acid sequences that encode for selectable markers in the target cells, so as to identify cells that have taken up the one or more polynucleotide sequences.

### Integration

The methods described herein provide for the integration of the circular nucleic acid into the genome of a human cell. In certain embodiments, the integration of the circular nucleic acid into the genome of the human cell involves an integrase. In certain embodiments, the integrase is a polypeptide. In certain embodiments, the integrase is a phiC31 integrase. In other embodiments, the integration involves a genome-editing enzyme. In certain embodiments, the genome-editing enzyme is a polypeptide. In certain embodiments, the genome-editing enzyme is Cas9 polypeptide, a zinc finger nuclease, a TALEN, or an enzymatically inactive type II CRISPR/Cas polypeptide. In other embodiments, the integration involves a RNA-guided endonuclease. In certain embodiments, the RNA-guided endonuclease is a polypeptide. In certain embodiments, the RNA-guided endonuclease is selected from a type II CRISPR/Cas polypeptide, a type V CRISPR/Cas polypeptide, and a type VI CRISPR/Cas polypeptide. In certain embodiments, the circular nucleic acid comprises a promoter and a reporter gene.

### Integrases

In certain embodiments, the integrases may be selected from, but not limited to, the following: phiC31, Bxb1, Cre, Flp, Tn3 resolvase, R4, or TP901-1. Integrases are also referred to as recombinases.

“Variant recombinases” include “mutant recombinases” are used interchangeably herein to refer to recombinase enzymes in which the native, wild-type recombinase gene found in the organism of origin has been mutated in one or more positions relative to a parent recombinase (e.g., in one or more nucleotides, which may result in alterations of one or more amino acids in the altered recombinase relative to a parent recombinase). “Parent recombinase” is used to refer to the nucleotide and/or amino acid sequence of the recombinase from which the altered recombinase is generated. The parent recombinase can be a naturally occurring enzyme (i.e., a native or wild-type enzyme) or a non-naturally occurring enzyme (e.g., a genetically engineered enzyme). The mutations present in an altered recombinase may comprise base substitutions, deletions, additions, and/or other rearrangements in the DNA sequence encoding the recombinase, and/or any combination of such mutations, either singly or in groups.

In certain embodiments, the methods involve one or more, such as two or more, three or more, four or more, or five or more integrases. For example, in certain embodiments, a first integrase is phiC31. In certain embodiments, a second integrase is Bxb1. PhiC31 and Bxb1 integrases used in the methods described herein include variant phiC31 and Bxb1 integrases that retain their activity that mediate site-specific recombination between specific DNA sequences recognized by the integrases.

Genome-Editing Enzymes

In certain cases, the insertion of the circular nucleic acid may be carried out by a targeted nuclease. One example of a targeted nuclease that may be used in the subject methods is a TAL Nuclease (“TALN”, TAL effector nuclease, or “TALEN”). A TALN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940, which is herein incorporated by reference. The most recognized example of a TALEN in the art is a fusion polypeptide of the FokI nuclease to a TAL effector DNA binding domain.

Another example of a targeted nuclease that finds use in the subject methods is a zinc finger nuclease or “ZFN”. ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include C₂H₂ zinc fingers, C₃H zinc fingers, and C₄ zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the FokI nuclease with a zinc finger DNA binding domain.

Another example of a targeted nuclease that finds use in the subject methods is a targeted Spo11 nuclease, a polypeptide comprising a Spo11 polypeptide having nuclease activity fused to a DNA binding domain, e.g. a zinc finger DNA binding domain, a TAL effector DNA binding domain, etc. that has specificity for a DNA sequence of interest. See, for example, U.S. Application No. 61/555,857, the disclosure of which is incorporated herein by reference.

Other non-limiting examples of targeted nucleases include naturally occurring and recombinant nucleases, e.g. CRISPR/Cas9, restriction endonucleases, meganucleases homing endonucleases, and the like. Examples of a CRISPER/Cas9 system may be found in, for example, Turan et al. (2016) and Geisinger et al. (2016), the disclosures of which are incorporated herein by reference.

Examples of RNA-guided endonucleases are CRISPR/Cas endonucleases (e.g., class 2 CRISPR/Cas endonucleases such as a type II, type V, or type VI CRISPR/Cas endonucleases). A suitable genome editing nuclease is a CRISPR/Cas endonuclease (e.g., a class 2 CRISPR/Cas endonuclease such as a type II, type V, or type VI CRISPR/Cas endonuclease). In some cases, a suitable RNA-guided endonuclease is a class 2 CRISPR/Cas endonuclease. In some cases, a suitable RNA-guided endonuclease is a class 2 type II CRISPR/Cas endonuclease (e.g., a Cas9 protein). In some cases, a genome targeting composition includes a class 2 type V CRISPR/Cas endonuclease (e.g., a Cpf1 protein, a C2c1 protein, or a C2c3 protein). In some cases, a suitable RNA-guided endonuclease is a class 2 type VI CRISPR/Cas endonuclease (e.g., a C2c2 protein; also referred to as a “Cas13a” protein). Also suitable for use is a CasX protein. Also suitable for use is a CasY protein.

In some cases, the genome-editing endonuclease is a Type II CRISPR/Cas endonuclease. In some cases, the genome-editing endonuclease is a Cas9 polypeptide. The Cas9 protein is guided to a target site (e.g., stabilized at a target site) within a target nucleic acid sequence (e.g., a chromosomal sequence or an extrachromosomal sequence, e.g., an episomal sequence, a minicircle sequence, a mitochondrial sequence, a chloroplast sequence, etc.) by virtue of its association with the protein-binding segment of the Cas9 guide RNA. In some cases, the Cas9 polypeptide used in a composition or method of the present disclosure is a Staphylococcus aureus Cas9 (saCas9) polypeptide. In some cases, the genome-editing endonuclease is a CasX or a CasY polypeptide. CasX and CasY polypeptides are described in Burstein et al. (2017) Nature 542:237.

Targeted nucleases may be used in pairs, with one targeted nuclease specific for one sequence of an integration site and the second targeted nuclease specific for a second sequence of an integration site. In the methods presented herein, any targeted nuclease(s) that are specific for the integration site of interest and promote the cleavage of an integration site may be used. The targeted nuclease(s) may be stably expressed by the cells. Alternatively, the targeted nuclease(s) may be transiently expressed by the cells, e.g. it may be provided to the cells prior to, simultaneously with, or subsequent to contacting the cells with the landing pad generating polynucleotide. If transiently expressed by the cells, the targeted nuclease(s) may be provided to cells as DNA, e.g. plasmid or vector, as described herein, e.g., by using transfection, nucleofection, or the like. Alternatively, targeted nuclease(s) may be provided to cells as mRNA encoding the targeted nuclease(s), e.g. using well-developed transfection techniques; see, e.g. Angel and Yanik (2010) PLoS ONE 5(7): e11756; Beumer et al. (2008) PNAS 105(50):19821-19826, and the commercially available TransMessenger® reagents from Qiagen, Stemfect™ RNA Transfection Kit from Stemgent, and TransIT®-mRNA Transfection Kit from Mirus Bio LLC. Alternatively, the targeted nuclease(s) may be provided to cells as a polypeptide. Such polypeptides may optionally be fused to a polypeptide domain that increases solubility of the product, and/or fused to a polypeptide permeant domain to promote uptake by the cell. The targeted nuclease(s) may be produced by eukaryotic cells or by prokaryotic cells, it may be further processed by unfolding, e.g. heat denaturation, DTT reduction, etc. and may be further refolded, using methods known in the art. It may be modified, e.g. by chemical derivatization or by molecular biology techniques and synthetic chemistry, e.g. to so as to improve resistance to proteolytic degradation or to optimize solubility properties or to render the polypeptide more suitable as a therapeutic agent.

Also suitable for use is an RNA-guided endonuclease with reduced enzymatic activity. Such an RNA-guided endonuclease is referred to as a “dead” RNA-guided endonuclease; for example, a Cas9 polypeptide that comprises certain amino acid substitutions such that it exhibits substantially no endonuclease activity, but such that it still binds to a target nucleic acid when complexed with a guide RNA, is referred to as a “dead” Cas9 or “dCas9.” In some cases, a “dead” Cas9 protein has a reduced ability to cleave both the complementary and the non-complementary strands of a double stranded target nucleic acid. For example, a “nuclease defective” Cas9 lacks a functioning RuvC domain (i.e., does not cleave the non-complementary strand of a double stranded target DNA) and lacks a functioning HNH domain (i.e., does not cleave the complementary strand of a double stranded target DNA). Such a Cas9 protein has a reduced ability to cleave a target nucleic acid (e.g., a single stranded or double stranded target nucleic acid) but retains the ability to bind a target nucleic acid. A Cas9 protein that cannot cleave target nucleic acid (e.g., due to one or more mutations, e.g., in the catalytic domains of the RuvC and HNH domains) is referred to as a “nuclease defective Cas9”, “dead Cas9” or simply “dCas9.” Other residues can be mutated to achieve the above effects (i.e. inactivate one or the other nuclease portions). As non-limiting examples, residues D10, G12, G17, E762, H840, N854, N863, H982, H983, A984, D986, and/or A987 of Streptococcus pyogenes Cas9 (or the corresponding amino acids of a Cas9 homolog) can be altered (i.e., substituted). In some cases, two or more of D10, E762, H840, N854, N863, and D986 of Streptococcus pyogenes Cas9 (or the corresponding amino acids of a Cas9 homolog) are substituted. In some cases, D10 and N863 of Streptococcus pyogenes Cas9 (or the corresponding amino acids of a Cas9 homolog) are substituted with Ala. Also, mutations other than alanine substitutions are suitable.

In some cases, the genome-editing endonuclease is an RNA-guided endonuclease (and it corresponding guide RNA) known as Cas9-synergistic activation mediator (Cas9-SAM). The RNA-guided endonuclease (e.g., Cas9) of the Cas9-SAM system is a “dead” Cas9 fused to a transcriptional activation domain (wherein suitable transcriptional activation domains include, e.g., VP64, p65, MyoD1, HSF1, RTA, and SET7/9) or a transcriptional repressor domain (where suitable transcriptional repressor domains include, e.g., a KRAB domain, a NuE domain, an NcoR domain, a SID domain, and a SID4X domain). The guide RNA of the Cas9-SAM system comprises a loop that binds an adapter protein fused to a transcriptional activator domain (e.g., VP64, p65, MyoD1, HSF1, RTA, or SET7/9) or a transcriptional repressor domain (e.g., a KRAB domain, a NuE domain, an NcoR domain, a SID domain, or a SID4X domain). For example, in some cases, the guide RNA is a single-guide RNA comprising an MS2 RNA aptamer inserted into one or two loops of the sgRNA; the dCas9 is a fusion polypeptide comprising dCas9 fused to VP64; and the adaptor/functional protein is a fusion polypeptide comprising: i) MS2; ii) p65; and iii) HSF1. See, e.g., U.S. Patent Publication No. 2016/0355797.

Contacting the cells with the polypeptide may occur in any culture media and under any culture conditions that promote the survival of the cells. For example, cells may be suspended in any appropriate nutrient medium, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors. Conditions that promote the survival of cells are typically permissive of nonhomologous end joining and homologous recombination.

Dual-Integrase Cassette Exchange System (DICE)

In certain embodiments, the method further includes inserting a polynucleotide sequence of interest into a genome of a human cell using DICE. Examples of methods of inserting a polynucleotide sequence into a genome of a human cell using DICE may be found in, for example, in PCT publication WO2015073703, the disclosure of which is incorporated herein by reference.

In certain embodiments, a method of inserting a polynucleotide sequence into a genome of a human cell includes inserting one or more polynucleotide sequences into a genome of a human cell, comprising: introducing into the human cell: a circular nucleic acid with the one or more polynucleotide sequences flanked by a first recombination site of a first integrase and a first recombination site of a second integrase; the first integrase; and the second integrase, wherein the human cell includes a second recombination site of the first integrase and a second recombination site of the second integrase at a locus in a chromosome; maintaining the human cell under conditions that facilitate recombination between first recombination site of the first integrase and the second recombination site of the first integrase and between the first recombination site of the second integrase and the second recombination site of the second integrase, wherein the introducing and maintaining results in insertion of the one or more polynucleotide sequences into the genome of the human cell at the locus. In certain embodiments, the first integrase is phiC31. In certain embodiments, the second integrase is Bxb1.

As used herein a “recombination site” or “recognition site” is a DNA sequence that serves a substrate for a recombinase so as to provide for unidirectional site-specific recombination. In general, the recombinases used in the invention involve two recognition sites, one that is positioned in the integration site (the site into which a nucleic acid is to be integrated) and another adjacent a nucleic acid of interest to be introduced into the integration site. The terms “recombinase” and “integrase” are used interchangeably herein.

As used herein, a phiC31 first recombination site and a phiC31 second recombination site refers to nucleic acid sequences that are recognized by the phiC31 integrase and used as a substrates for recombination between the nucleic acid sequences. In certain cases, the phiC31 first recombination site may be phiC31 attB and the phiC31 second recombination site may be phiC31 attP. In other cases, the phiC31 first recombination site may be phiC31 attP and the phiC31 second recombination site may be phiC31 attB. phiC31 attP and phiC31 attB are DNA sequences specifically recognized by phiC31 integrase.

As used herein, a Bxb1 first recombination site and a Bxb1 second recombination site refers to recognition sequences that are recognized by the Bxb1 integrase and used as a substrates for recombination between the recognition sequences. In certain cases, the Bxb1 first recombination site may be Bxb1 attB and the Bxb1 second recombination site may be Bxb1 attP. In other cases, the Bxb1 first recombination site may be Bxb1 attP and the Bxb1 second recombination site may be Bxb1 attB. Bxb1 attP and Bxb1 attB are DNA sequences specifically recognized by Bxb1 integrase.

AttB and attP sites are general names for the recombination site pairs that are recognized by and recombined by bacteriophage integrases, such as, phiC31 and Bx1. In general, the sequence of attB and attP sites are different from each other and further the sequences of attB and attP sites pairs that recognized and recombined by different integrases are different.

A “recognition site” is a DNA sequence that serves a substrate for a wild-type or variant recombinase so as to provide for unidirectional site-specific recombination. In general, the recombinases used in the invention involve two recognition sites, one that is positioned in the integration site (the site into which a nucleic acid is to be integrated) and another adjacent a nucleic acid of interest to be introduced into the integration site. For example, the recognition sites for phage integrases phiC31 and Bxb1 are generically referred to as attB and attP. Recognition sites can be native or altered relative to a native sequence. Use of the term “recognize” in the context of a recombinase “recognizes” a recognition sequence, is meant to refer to the ability of the recombinase to interact with the recognition site and facilitate site-specific recombination.

In certain cases, the phiC31 attB and attP site sequences and the phiC31 integrase may be as provided in U.S. Pat. Nos. 8,304,233 or 8,420,395, the entire disclosures of which are herein incorporated by reference.

In certain cases, the Bxb1 attB and attP site sequences and the Bxb1 integrase may be as provided in US 20110136237 and US 20080020465, the entire disclosures of which are herein incorporated by reference.

### Nucleic Acid Compositions

As noted above, a circular nucleic acid is provided in the present disclosure. A circular nucleic acid may include a polynucleotide sequence for FST, alone, or in combination with additional polynucleotides such as DYSF, CAPN3, SGCA, and/or MSX1. In certain embodiments, a resistance gene may be replaced to include an additional sequence for a polynucleotide. In other embodiments, the puromycin resistance gene is replaced to include an additional polynucleotide sequence, e.g., a sequence encoding DYSF. In addition, the circular nucleic acid may include an expression cassette, a reporter gene, a resistance gene, and/or a detectable signal.

In certain cases, the reporter gene may be an “imaging marker”. An imaging marker may a non-cytotoxic agent that can be used to locate and, optionally, visualize cells, e.g. cells that have been targeted by nucleic acid compositions of the subject application. An imaging marker may require the addition of a substrate for detection, e.g., horseradish peroxidase (HRP), β-galactosidase, luciferase, and the like. Alternatively, an imaging marker may provide a detectable signal that does not require the addition of a substrate for detection, e.g. a fluorophore or chromophore dye, e.g. Alexa Fluor 488® or Alexa Fluor 647®, or a protein that comprises a fluorophore or chromophore, e.g. a fluorescent protein. As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak in the emission spectrum at 510 nm or about 510 nm. A variety of FPs that emit at various wavelengths are known in the art. FPs of interest include, but are not limited to, a green fluorescent protein (GFP), yellow fluorescent protein (YFP), orange fluorescent protein (OFP), cyan fluorescent protein (CFP), blue fluorescent protein (BFP), red fluorescent protein (RFP), far-red fluorescent protein, or near-infrared fluorescent protein and variants thereof.

By a “selection marker” or “selectable marker” it is meant an agent that can be used to select cells, e.g., cells that have been targeted by nucleic acid compositions of the subject application. In some instances, the selection may be positive selection; that is, the cells are isolated from a population, e.g. to create an enriched population of cells comprising the genetic modification. In other instances, the selection may be negative selection; that is, the population is isolated away from the cells, e.g. to create an enriched population of cells that do not comprise the genetic modification. Any convenient selectable marker may be employed, for example, a drug selectable marker, e.g. a marker that prevents cell death in the presence of drug, a marker that promotes cell death in the presence of drug, an imaging marker, etc.; an imaging marker that may be selected for using imaging technology, e.g. fluorescence activated cell sorting; a polypeptide or peptide that may be selected for using affinity separation techniques, e.g. fluorescence activated cell sorting, magnetic separation, affinity chromatography, “panning” with an affinity reagent attached to a solid matrix, etc.; and the like.

### Target Cells of Interest

The subject methods may be employed to insert into and/or bind target cells (e.g., for visualization, for collecting and/or analyzing, etc.) in mitotic or post-mitotic cells in vivo and/or ex vivo and/or in vitro. A mitotic and/or post-mitotic cell of interest in the disclosed methods may include a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal, a cell from a rodent, a cell from a human, etc.).

Any type of cell may be of interest (e.g. a stem cell, e.g. an embryonic stem (ES) cell, an induced pluripotent stem (iPS) cell, a germ cell; a somatic cell, e.g. a fibroblast, a hematopoietic cell, a neuron, a muscle cell, a bone cell, a hepatocyte, a pancreatic cell; an in vitro or in vivo embryonic cell of an embryo at any stage, e.g., a 1-cell, 2-cell, 4-cell, 8-cell, etc. stage zebrafish embryo; etc.). Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e. splitting of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. Target cells are in many embodiments unicellular organisms, or are grown in culture.

If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, phosphate-buffered saline (PBS), Hank's balanced salt solution, etc., conveniently supplemented with fetal calf serum or other naturally occurring factors, in conjunction with an acceptable buffer at low concentration, generally from 5-25 mM. Convenient buffers include Hank's balanced salt solution, HEPES, phosphate buffers, lactate buffers, etc. The cells may be used immediately, or they may be stored, frozen, for long periods of time, being thawed and capable of being reused. In such cases, the cells will usually be frozen in 10% or more DMSO, 50% or more serum, and about 40% buffered medium, or some other such solution as is commonly used in the art to preserve cells at such freezing temperatures, and thawed in a manner as commonly known in the art for thawing frozen cultured cells.

Method of Delivering Polynucleotides into a Human Cell of a Target Tissue

In certain embodiments, a method of delivering one or more polynucleotides into a human cell of a target tissue includes: creating an opening in the subject; introducing the circular nucleic acid including the one or more polynucleotide sequences into the target tissue; and applying electroporation to the target tissue. In certain embodiments, a method of delivering one or more polynucleotides into a human cell of a target tissue includes: positioning a device in contact with a subject to restrict blood flow to a limb; creating an opening in the subject to expose a vessel; introducing a circular nucleic acid including the one or more polynucleotides into the vessel; releasing the device; and closing the opening. Examples of methods of delivering a polynucleotide into a human cell of a target tissue may be found in, for example, Portlock et al. (2006), Bertoni et al. (2006), Wolf et al. (1990), and Hagstrom et al. (2004), the disclosures of which are incorporated herein by reference. In certain embodiments, the circular nucleic acid includes a promoter and a reporter gene.

In certain embodiments, the step of introducing a circular nucleic acid and a polypeptide into the human cell may be carried out by any method known in the art. For example, a nucleic acid may be introduced into a human cell by injection (into the nucleus or cytoplasm), transfection, viral infection, nucleofection, electroporation, calcium chloride transfection, and lipofection, and the like. Nucleic acid can be introduced as naked nucleic acid, as nucleic acid complexed with an agent such as a liposome or a stabilizing agent and the like.

### Intramuscular Injection

For methods of delivering one or more polynucleotides into a human cell of a target tissue, in some cases, the one or more polynucleotides are provided as a nucleic acid (e.g., an mRNA, a DNA, a plasmid, an expression vector, etc.) that encodes the one or more polypeptides. In certain cases, the one or more polynucleotides are provided directly as a protein. The one or more polynucleotides may be introduced into a cell by any convenient method. In certain cases, an effective amount of circular nucleic acid is delivered to a target tissue. Examples of methods of intramuscular injection may be found in, for example, Portlock et al. (2006) and Bertoni et al. (2006), the disclosures of which are incorporated herein by reference.

### Electroporation

In certain embodiments, electroporation of the target tissue is applied after injection. Electroporation is the phenomenon in which permeability of the cell membrane to ions and macromolecules is increased by exposing the cell to short (microsecond to millisecond) high voltage electric pulses. Several hundred volts across a distance of several millimeters are typically used in this process. A device with a two-needle electrode array may be applied to the target tissue to establish the requisite voltage after injection. Examples of methods of electroporation may be found in, for example, Bertoni et al. (2006).

### Device to Restrict Blood Flow to a Limb

In certain embodiments, a device in contact with a subject is used to restrict blood flow to a limb. A device may be applied to the distal limb to establish a bloodless field during surgery by application of direct pressure against a particular portion of the distal limb to regulate blood flow through the vessels within the limb.

In certain embodiments, the device used is a tourniquet. A tourniquet, in accordance with the present invention, comprises a strap with fastening material at end portions thereof for securing opposite ends of the strap together in an operative position encircling a limb or a part of a patient's body to restrict blood flow. Pressure is applied directly against a portion of the limb and indirectly against the opposite portion of the limb or part lying adjacent the strap, i.e., creating a sandwiching pressure.

In other embodiments, pneumatic cuffs may be used that utilize a strain gauge to detect the circumference of the patient's limb while blood flow is restricted by a pressure cuff. In certain embodiments, any suitable means to attain the desired effect of restricting blood flow may be used, including a compression band, a blood flow restriction band, an occlusion cuff, a mass casualty tourniquet, a silicon ring auto-transfusion tourniquet, a combat application tourniquet, or a cuff stabilizer.

### Vascular Injection

In certain cases, an effective amount of circular nucleic acid is delivered to contact at least a target tissue. In some cases, the circular nucleic acid is delivered at a rate of 8 ml/minutes to allow the circular nucleic acid to contact at least the target tissue, before applying a pressure for 1 minute. Examples of methods of vascular injection may be found in, for example, Hagstrom et al. (2004), the disclosures of which are incorporated herein by reference.

To induce uptake of nucleic acid into the surrounding tissue, the circular nucleic acid must be delivered at a rate of 1 ml/minute or more, such as 2 ml/minutes, 5 ml/minutes, 6 ml/minutes, 8 ml/minutes, 10 ml/minutes, 15 ml/minutes, 20 ml/minutes, 25 ml/minutes, or 30 ml/minutes. The rate of delivery as described herein my range from about 1 ml/minute to 50 ml/minutes, inclusive, such as 1 ml/minute to 40 ml/minutes, 1 mil/minute to 35 ml/minutes, 1 ml/minute to 30 ml/minutes, 1 ml/minute to 25 ml/minutes, 1 ml/minute to 20 ml/minutes, 1 ml/minute to 10 ml/minutes, or 1 ml/minute to 5 ml/minutes, inclusive.

In certain embodiments, the injections are performed in an anterograde direction (i.e., with the blood flow). In certain embodiments, the injections are performed against the blood flow. In certain embodiments, repeat deliveries of a circular nucleic acid may be utilized. Repeat deliveries of a circular nucleic acid may be performed continuously or intermittently. The time between each delivery of circular nucleic acids may be about 1 day to about 6 months, e.g., 2 days, 4 days, 6 days, 1 week, 2 weeks, 3 weeks, 4 weeks, 1 month, 2 months, 3 months, 4 months, 5 months, 6 months, or any other period from about 1 day to about 6 months, which may be repeated with a frequency of about every day to about every 4 days, e.g., every 1.5 days, every 2 days, every 3 days, or any other frequency from about every day to about every four days. The agent(s) may be provided to the subject cells one or more times, e.g. one time, twice, three times, or more than three times, and the cells allowed to incubate with the nucleic acids for some amount of time following each contacting event e.g. 1-4 weeks.

In certain embodiments, the dose of delivery changes between each repeat delivery. The dose of delivery may increase or decrease 1.5 times as much to about 10 times as much, e.g., 1.5 times, 2 times, 3 times, 4 times, 5 times, 6 times, 7 times, 8 times, 9 times, 10 times, or any other amount from about 1.1 times to about 10 times.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

### Genetically Modified Cells

The present disclosure provides a genetically modified mammalian host cell, where the mammalian host cell is genetically modified with: a) a circular nucleic acid as described above; or b) a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence.

In certain embodiments, a subject genetically modified host cell is in vitro. In some embodiments, a subject genetically modified host cell is in vivo. In some embodiments, a subject genetically modified host cell is a prokaryotic cell or is derived from a prokaryotic cell. In some embodiments, a subject genetically modified host cell is a bacterial cell or is derived from a bacterial cell. In some embodiments, a subject genetically modified host cell is an archaeal cell or is derived from an archaeal cell. In some embodiments, a subject genetically modified host cell is a eukaryotic cell or is derived from a eukaryotic cell. In some embodiments, a subject genetically modified host cell is a plant cell or is derived from a plant cell. In some embodiments, a subject genetically modified host cell is an animal cell or is derived from an animal cell. In some embodiments, a subject genetically modified host cell is an invertebrate cell or is derived from an invertebrate cell. In some embodiments, a subject genetically modified host cell is a vertebrate cell or is derived from a vertebrate cell. In some embodiments, a subject genetically modified host cell is a mammalian cell or is derived from a mammalian cell. In some embodiments, a subject genetically modified host cell is a rodent cell or is derived from a rodent cell. In some embodiments, a subject genetically modified host cell is a human cell or is derived from a human cell.

In certain embodiments, the genetically modified human cell may be a human cell that includes a polynucleotide sequence of interest at a locus flanked by a recombined recognition sequence of phiC31 integrase and a recombined recognition sequence of Bxb1 integrase. In certain embodiments, the recipient cell that includes insertion of the phiC31 second recombination site and the Bxb1 second recombination site at a locus may be isolated or enriched prior to the step of introducing into the human cell: a circular nucleic acid comprising the polynucleotide sequence flanked by a phiC31 first recombination site and a Bxb1 first recombination site; a phiC31 integrase; and a Bxb1 integrase.

In certain embodiments, the human cell that is genetically modified to include a polynucleotide sequence of interest may be isolated or enriched. Using the method disclosed herein, genetically modified cells including a polynucleotide sequence of interest may be enriched relative to cells that do not contain one or more polynucleotide sequences of interest, e.g., at least 2 fold, at least 3 fold, at least 5 fold, at least 10 fold, at least 20 fold, at least 30 fold, at least 40 fold, at least 50 fold, at least 100 fold, or more.

Separation of genetically modified cells typically relies upon the expression of a selectable marker that is co-integrated into the target locus. By a “selectable marker” it is meant an agent that can be used to select cells, e.g. cells that have been targeted by compositions of the subject application. In some instances, the selection may be positive selection; that is, the cells are isolated from a population, e.g. to create an enriched population of cells comprising the genetic modification. In other instances, the selection may be negative selection; that is, the population is isolated away from the cells, e.g. to create an enriched population of cells that do not comprise the genetic modification. Separation may be by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells.

Cell compositions that are highly enriched for cells comprising the polynucleotide sequence of interest are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a human subject for purposes such as gene therapy, e.g. to treat a disease, or as an antiviral, antipathogenic, or anticancer therapeutic. The subject may be a neonate, a juvenile, or an adult.

The genetically modified cells of the present invention may be formulated into cell compositions that are pharmaceutical compositions that include a pharmaceutically acceptable carrier. Examples of pharmaceutically acceptable carriers include saline, buffers, diluents, fillers, salts, stabilizers, solubilizers, cell culture medium, and other materials which are well known in the art. In some embodiments, the formulations are free of detectable DMSO (dimethyl sulfoxide).

Any human cell's genome may be modified by the nucleic acid compositions and methods described herein. For example, the cell may be a meiotic cell, a mitotic cell, or a post-mitotic cell. Mitotic and post-mitotic cells of interest in these embodiments include pluripotent stem cells, e.g. ES cells, iPS cells, and embryonic germ cells; and somatic cells, e.g. fibroblasts, hematopoietic cells, neurons, muscle cells, bone cells, vascular endothelial cells, gut cells, and the like, and their lineage-restricted progenitors and precursors.

Cells may be modified in vitro or in vivo. If modified in vitro, cells may be from established cell lines or they may be primary cells, where “primary cells”, “primary cell lines”, and “primary cultures” are used interchangeably herein to refer to cells and cells cultures that have been derived from a subject, such as, a human subject and either modified without significant additional culturing, i.e. modified “ex vivo”, e.g. for return to the subject, or allowed to grow in vitro for a limited number of passages, i.e. splittings, of the culture. For example, primary cultures are cultures that may have been passaged 0 times, 1 time, 2 times, 4 times, 5 times, 10 times, or 15 times. Typically, the primary cell lines of the present disclosure are maintained for fewer than 10 passages in vitro.

If the cells are primary cells, they may be harvested from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, nervous system, etc., are most conveniently harvested by biopsy. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The genetically modified cells of the present disclosure may be provided to the human subject alone or with a suitable substrate or matrix, e.g., to support their growth and/or organization in the tissue to which they are being transplanted. Usually, at least 1×10³ cells may be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, intramuscular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint; by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference).

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

### Utility

The nucleic acid compositions, cell compositions, and methods disclosed herein find use in any in vitro or in vivo application in which it is desirable to deliver one or more polynucleotides into a human cell of a target tissue and express one or more polynucleotide sequences/genes of interest in a human cell. In some cases, the therapy is non-immunogenic and may be re-administered multiple times. In some cases, the therapy is long-lasting especially if the nucleic acid becomes integrated into the chromosomes at transcriptionally active locations. In some cases, the therapy involves sequence-specific integration of a polynucleotide sequence into a genome of a human cell.

For example, the subject methods and compositions may be used to treat a disorder, a disease, or medical condition in a subject. Towards this end, the one or more genes of interest to be integrated into a cellular genome may include a gene that encodes for a therapeutic agent. By a “therapeutic agent” it is meant an agent, e.g. siRNA, shRNA, miRNA, CRISPRi agents, peptide, polypeptide, suicide gene, etc., that has a therapeutic effect upon a cell or an individual, for example, that promotes a biological process to treat a medical condition, e.g. a disease or disorder. The terms “individual,” “subject,” “host,” and “patient,” are used interchangeably herein and refer to any human subject for whom diagnosis, treatment, or therapy is desired. The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. In some cases, the treated symptom may include altering the number of centronucleated fibers. In some other cases, the number of centronucleated fibers is reduced. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

Examples of therapeutic agents that may be integrated into a cellular genome using the subject methods and compositions include agents, i.e., siRNAs, shRNAs, miRNAs, CRISPRi agents, peptides, or polypeptides, which alter cellular activity. Other examples of therapeutic agents that may be integrated using the subject methods and compositions include suicide genes, i.e., genes that promote the death of cells in which the gene is expressed. Non-limiting examples of suicide genes include genes that encode a peptide or polypeptide that is cytotoxic either alone or in the presence of a cofactor, e.g., a toxin such as abrin, ricin A, Pseudomonas exotoxin, cholera toxin, diphtheria toxin, Herpes Simplex Thymidine Kinase (HSV-TK); genes that promote apoptosis in cells, e.g. Fas, caspases (e.g. inducible Caspase9) etc.; and genes that target a cell for ADCC or CDC-dependent death, e.g. CD20.

In some instances, the therapeutic agent alters the activity of the cell in which the agent is expressed. In other words, the agent has a cell-intrinsic effect. For example, the agent may be an intracellular protein, transmembrane protein or secreted protein that, when expressed in a cell, will substitute for, or “complement”, a mutant protein in the cell. In other instances, the therapeutic agent alters the activity of cells other than cells in which the agent is expressed. In other words, the agent has a cell-extrinsic effect. For example, the integrated gene of interest may encode a cytokine, chemokine, growth factor, hormone, antibody, or cell surface receptor that modulates the activity of other cells.

The subject methods and compositions may be applied to any disease, disorder, or natural cellular process that would benefit from modulating cell activity by integrating a gene of interest. For example, the subject agents and methods find use in treating genetic disorders. Any genetic disorder that results from a single gene defect may be treated by the subject compositions and methods, including, for example, muscular dystrophies, e.g., Duchenne muscular dystrophy, limb girdle muscular dystrophy A, B, C, D; muscular conditions, e.g., sarcopenia, muscle injury, and cachexia; and neurodegenerative diseases, e.g., Parkinson's' disease, hemophilia, adenosine deaminase deficiency, sickle cell disease, X-Linked Severe Combined Immunodeficiency (SCID-X1), thalassemia, cystic fibrosis, alpha-1 anti-trypsin deficiency, diamond-blackfan anemia, Gaucher's disease, growth hormone deficiency, and the like. As another for example, the subject methods may be used to in medical conditions and diseases in which it is desirable to ectopically express a therapeutic agent, e.g. siRNA, shRNA, miRNA, CRISPRi agent, peptide, polypeptide, suicide gene, etc., to promote tissue repair, tissue regeneration, or protect against further tissue insult, e.g. to promote wound healing; promote the survival of the cell and/or neighboring cells, e.g. in degenerative disease, e.g. neurodegenerative disease, kidney disease, liver disease, etc.; prevent or treat infection, etc. Examples of muscular dystrophy may be found in, for example, Pichavant et al. (2017), the disclosure of which is incorporated herein by reference.

As one non-limiting example, the subject methods may be used to integrate a gene encoding a neuroprotective factor, e.g. a neurotrophin (e.g. NGF, BDNF, NT-3, NT-4, CNTF), Kifap3, Bcl-xl, Crmpl, Chkβ, CALM2, Caly, NPG11, NPT1, Eef1a1, Dhps, Cd151, Morf412, CTGF, LDH-A, At11, NPT2, Ehd3, Cox5b, Tubala, γ-actin, Rpsa, NPG3, NPG4, NPG5, NPG6, NPG7, NPG8, NPG9, NPG10, etc., into the genome of neurons, astrocytes, oligodendrocytes, or Schwann cells at a locus that is active in those particular cell types (for example, for neurons, the neurofilament (NF), neuro-specific enolase (NSE), NeuN, or Map2 locus; for astrocytes, the GFAP or S 100B locus; for oligodendrocytes and Schwann cells, the GALC or MBP locus). Such methods may be used to treat nervous system conditions and to protect the CNS against nervous system conditions, e.g. neurodegenerative diseases, including, for example, e.g. Parkinson's Disease, Alzheimer's Disease, Huntington's Disease, Amyotrophic Lateral Sclerosis (ALS), Spielmeyer-Vogt-Sjögren-Batten disease (Batten Disease), Frontotemporal Dementia with Parkinsonism, Progressive Supranuclear Palsy, Pick Disease, prion diseases (e.g. Creutzfeldt-Jakob disease), Amyloidosis, glaucoma, diabetic retinopathy, age related macular degeneration (AMD), and the like); neuropsychiatric disorders (e.g. anxiety disorders (e.g. obsessive compulsive disorder), mood disorders (e.g. depression), childhood disorders (e.g. attention deficit disorder, autistic disorders), cognitive disorders (e.g. delirium, dementia), schizophrenia, substance related disorders (e.g. addiction), eating disorders, and the like); channelopathies (e.g. epilepsy, migraine, and the like); lysosomal storage disorders (e.g. Tay-Sachs disease, Gaucher disease, Fabry disease, Pompe disease, Niemann-Pick disease, Mucopolysaccharidosis (MPS) & related diseases, and the like); autoimmune diseases of the CNS (e.g. Multiple Sclerosis, encephalomyelitis, paraneoplastic syndromes (e.g. cerebellar degeneration), autoimmune inner ear disease, opsoclonus myoclonus syndrome, and the like); cerebral infarction, stroke, traumatic brain injury, and spinal cord injury.

In certain embodiments, the subject methods find use in treating muscular dystrophy, such as, Duchenne muscular dystrophy, limb girdle muscular dystrophy 2B, and limb girdle muscular dystrophy 2D. When used to treat Duchenne muscular dystrophy in a subject, the polynucleotide sequence inserted into the genome of the human cell may include a polynucleotide encoding dystrophin, such as, full length dystrophin or functional fragments or a functional variant thereof. In certain cases, a subject having limb girdle muscular dystrophy 2B may be treated using the subject methods, where the polynucleotide sequence inserted into the genome of the human cell may encode for DYSF or a functional fragment or a functional variant thereof. In certain cases, a subject having limb girdle muscular dystrophy 2D may be treated using the subject methods, where the polynucleotide sequence inserted into the genome of the human cell may encode SGCA or a functional fragment or a functional variant thereof. In certain cases, the human cell may be an iPS cell derived from a cell of the subject being treated.

The subject methods and compositions may be applied as a means to create regenerative stem cells within a target tissue. As one non-limiting example, gene therapy with the MSX1 gene may be used bring about localized de-differentiation in muscle fibers. The MSX1 protein interacts with TATA-box binding protein and may repress transcription of target genes, such as MyoD, a regulator of muscle differentiation. In some cases, muscle stem cells may be generated in situ, where they carry out muscle repair. In the context of a dystrophic muscle, MSX1 may be introduced by gene therapy alone, or along with the appropriate therapeutic gene for a particular form of muscular dystrophy, such as dystrophin for Duchenne muscular dystrophy. For example, a gene cassette may be introduced that included genes for CAPN3, SGCA, DYSF and/or MSX1, the latter under control of an inducible promoter to regulate gene expression in a temporal fashion.

Therapeutic compositions comprising for use in altering a phenotypic characteristic of muscular dystrophy, treating muscular dystrophy, and/or alleviating a symptom of muscular dystrophy are also provided. Such compositions typically comprise a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence and a pharmaceutically acceptable carrier.

Various pharmaceutical compositions and techniques for their preparation and use are known to those of skill in the art in light of the present disclosure. For a detailed listing of suitable pharmacological compositions and techniques for their administration one may refer to the detailed teachings herein, which may be further supplemented by texts such as Remington's Pharmaceutical Sciences, 17th ed. 1985; Brunton et al., “Goodman and Gilman's The Pharmacological Basis of Therapeutics,” McGraw-Hill, 2005; University of the Sciences in Philadelphia (eds.), “Remington: The Science and Practice of Pharmacy,” Lippincott Williams & Wilkins, 2005; and University of the Sciences in Philadelphia (eds.), “Remington: The Principles of Pharmacy Practice,” Lippincott Williams & Wilkins, 2008.

Other examples of how the subject methods may be used to treat medical conditions are disclosed elsewhere herein, or would be readily apparent to the ordinarily skilled artisan. The subject methods may also be used to deliver, via muscle, secreted proteins (e.g., growth factors and clotting factors) that are needed elsewhere in a subject's body.

As discussed above, any gene of interest may be integrated into a target locus, for example, any gene encoding an siRNA, shRNA, miRNA, CRISPRi element, peptide, or polypeptide may be integrated. Additionally, as discussed above, more than one gene of interest may be integrated, for example, two or more genes of interest may be integrated, three or more genes may be integrated, four or more genes may be integrated, e.g. five or more genes may be integrated. Thus, for example, in an embodiment where the genetically modified cell is a ES or PS or iPS cell, the one of more polynucleotide sequences/genes of interest may be transcription factors that promote the differentiation of the ES or PS or iPS cell into a particular cell lineage. For example, the subject methods may be used to convert iPS generated from a somatic cell isolated from a subject in need of a particular cell type into the particular cell type. Since the cells to be transplanted into the subject are derived from the subject's cells, any immune response to the transplanted cells may be reduced or avoided.

Integrating one or more genes of interest into genomic DNA such that it is expressed in cell finds use in many fields, including, for example, gene therapy, and research. For example, such modifications are therapeutically useful, e.g. to treat a genetic disorder by complementing a genetic mutation in a subject with a wild-type copy of the gene; to promote naturally occurring processes, by promoting/augmenting cellular activities (e.g. promoting wound healing for the treatment of chronic wounds or prevention of acute wound or flap failure, by augmenting cellular activities associated with wound healing); to modulate cellular response (e.g. to treat diabetes mellitus, by providing insulin); to express antiviral, antipathogenic, or anticancer therapeutics in subjects, e.g. in specific cell populations or under specific conditions, etc. Other uses for such genetic modifications include in the induction of induced pluripotent stem cells (iPSCs), e.g. to produce iPSCs from an individual for diagnostic, therapeutic, or research purposes; in the production of genetically modified cells, for example in manufacturing for the large scale production of proteins by cells for therapeutic, diagnostic, or research purposes.

### Kits

The present disclosure provides kits for carrying out a subject method. A subject kit can include one or more of (e.g., two or more, three or more, four or more, or all five): a circular nucleic acid comprising one or more polynucleotide sequences, wherein the one or more polynucleotide sequences comprise at least an FST gene sequence and one of a CAPN3 gene sequence, an SGCA gene sequence, or a DYSF gene sequence; an expression vector that encodes a polypeptide; a nucleic acid encoding an integrase; an integrase; or a nucleic acid encoding a genome-editing enzyme, all of which are described in detail above.

In some embodiments, the subject kits may have a combination of nucleic acid compositions as described herein, or recipient cells as described herein, or both. Reagents of interest may include polynucleotide compositions, e.g. a target nuclease or pair of targeted nucleases specific for the target site in the locus; reagents for selecting cells genetically modified with the integrated gene of interest; and positive and negative control vectors or cells comprising integrated positive and/or negative control sequences for use in assessing the efficacy donor polynucleotide compositions in cells, etc.

A subject kit can further include one or more additional reagents, where such additional reagents can be selected from: a buffer; a wash buffer; a control reagent; a control expression vector or polynucleotide; and the like. Components of a subject kit can be in separate containers; or can be combined in a single container.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure, numbered 1-21 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of altering a phenotypic characteristic of muscular dystrophy, comprising:

introducing into the human cell a circular nucleic acid comprising one or more polynucleotide sequences, wherein the one or more polynucleotide sequences comprise at least a follistatin gene sequence and one of a calpain 3 (CAPN3) gene sequence, an alpha-sarcoglycan (SGCA) gene sequence, or a dysferlin (DYSF) gene sequence; and

expressing the one or more polynucleotide sequences in the circular nucleic acid in the human cell, wherein the expressing results in altering the phenotypic characteristic of muscular dystrophy.

Aspect 2. A method of treating muscular dystrophy, comprising:

introducing into the human cell a circular nucleic acid comprising one or more polynucleotide sequences, wherein the one or more polynucleotide sequences comprise at least a follistatin gene sequence and one of a calpain 3 (CAPN3) gene sequence, an alpha-sarcoglycan (SGCA) gene sequence, or a dysferlin (DYSF) gene sequence; and

expressing the one or more polynucleotide sequences in the circular nucleic acid in the human cell, wherein the expressing results in treating muscular dystrophy.

Aspect 3. A method of alleviating a symptom of muscular dystrophy, comprising:

introducing into the human cell a circular nucleic acid comprising one or more polynucleotide sequences, wherein the one or more polynucleotide sequences comprise at least a follistatin gene sequence and one of a calpain 3 (CAPN3) gene sequence, an alpha-sarcoglycan (SGCA) gene sequence, or a dysferlin (DYSF) gene sequence; and

expressing the one or more polynucleotide sequences in the circular nucleic acid in the human cell, wherein the expressing results in alleviating the symptom of muscular dystrophy.

Aspect 4. The method of any one of the preceding aspects, wherein the introducing into the human cell provides for integration of the circular nucleic acid into a genome of the human cell.

Aspect 5. The method of aspect 4, wherein the integration of the circular nucleic acid comprises an integrase.

Aspect 6. The method of aspect 5, wherein the integrase is encoded on a nucleic acid expression vector.

Aspect 7. The method of aspect 5, wherein the integrase is a polypeptide.

Aspect 8. The method of aspect 5, wherein the integrase is a phiC31 integrase.

Aspect 9. The method of aspect 4, wherein the integration of the circular nucleic acid comprises a genome-editing enzyme.

Aspect 10. The method of aspect 9, wherein the genome-editing enzyme is encoded on a nucleic acid expression vector.

Aspect 11. The method of aspect 9, wherein the genome-editing enzyme is a polypeptide.

Aspect 12. The method of aspect 9, wherein the genome-editing enzyme is a Cas9 polypeptide, a zinc finger nuclease, a TALEN, or an enzymatically inactive type II CRISPR/Cas polypeptide.

Aspect 13. The method of aspect 4, wherein the wherein the integration of the circular nucleic acid comprises an RNA-guided endonuclease

Aspect 14. The method of aspect 13, wherein the RNA-guided endonuclease is encoded on a nucleic acid expression vector.

Aspect 15. The method of aspect 13, wherein the RNA-guided endonuclease is a polypeptide.

Aspect 16. The method of any one of the preceding aspects, further comprising delivering one or more polynucleotides into a human cell of a target tissue, comprising:

creating an opening in a subject to expose the target tissue;

introducing the circular nucleic acid comprising the one or more polynucleotide sequences into the target tissue; and

applying electroporation to the target tissue.

Aspect 17. The method of any one of the preceding aspects, further comprising delivering one or more polynucleotides into a human cell of a target tissue, comprising:

positioning a device in contact with a subject to restrict blood flow to a limb;

creating an opening in the subject to expose a vessel;

introducing the circular nucleic acid comprising the one or more polynucleotides into the vessel;

applying a pressure;

releasing the device; and

closing the opening.

Aspect 18. The method of any one of the preceding aspects, wherein the circular nucleic acid comprises a promoter and a reporter gene.

Aspect 19. The method of any one of the preceding aspects, wherein the one or more polynucleotides encode a polypeptide.

Aspect 20. The method of aspect 19, wherein the polypeptide is a transcription factor.

Aspect 21. The method of any one of the preceding aspects, wherein the human cell is a muscle cell.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); i.m., intramuscular(ly); i.p., intraperitoneal(ly); s.c., subcutaneous(ly); and the like.

### Materials and Methods

### Plasmids and DNA

The therapeutic plasmid carrying the dysferlin coding sequence, pKLD-CAG-DYSF-CPL, was constructed as described in Turan et al. (2016). Briefly, the plasmid contained the human dysferlin cDNA cloned into the pKLD-CAG backbone, along with a gene expression cassette encoding mCherry, puromycin resistance, and firefly luciferase connected by p2A skipping peptides. To construct a derivative that also encoded follistatin, the puromycin resistance gene in pKLD-CAG-DYSF-CPL was replaced with the coding sequence for the 344-amino acid isoform of human follistatin, synthesized as a gBlock gene fragment (Integrated DNA Technologies, Coralville, Iowa), using the AgeI restriction enzyme site. Plasmid DNA used for animal injections was purified with an endotoxin-free maxi- or giga-prep kit (Macherey-Nagel, Duren, Germany), following the manufacturer's instructions.

### Mice

Dysferlin-null A/J mice that were crossed onto the C57Bl/6 background to make the Bl/AJ strain were utilized. See, e.g., Ho et al. (2004) and Lostal et al. (2010). Bl/AJ mice (B6.A-Dysfprmd/GeneJ, stock number 012767) were provided by the Jackson Laboratory (Bar Harbor, Me.) from a colony maintained by the Jain Foundation. To obtain immune-deficient mice, the Bl/AJ mice were crossed with NRG mice that were deficient in B, T, and NK cells. NRG mice (NOD.Cj-Rag1tm1Mom Il2rgtm1Wjl/Szj, stock number 007799) were purchased from Jackson Laboratory. The resulting B1AJ/NRG mice were characterized in detail and donated to Jackson Labs as stock number 029663 (NOD.Cg-Rag1tm1MomDysfprmdIl2rgtm1Wjl/McalJ).

### Vascular Injection

Plasmid DNA was injected into mouse hind limbs using a vascular delivery procedure. See, e.g., Hagstrom et al (2004). Mice were anesthetized with a ketamine-xylazine cocktail (K: 10 mg/ml, X: 1 mg/ml), a tourniquet was placed at the proximal joint of the leg, and a small incision was made near the ankle joint. 400 μg of plasmid DNA in 1 ml Hank's balanced salt solution (HBSS; ThermoFisher Scientific, Waltham, Mass.) or 1 ml HBSS buffer alone were injected into the saphenous vein using a 10 ml Luer-Lok syringe (Becton Dickinson, Franklin Lakes, N.J.) attached to a 33G needle (TSK Laboratories, Tochigi, Japan) at a rate of 8 ml/minute using a Legato 210 syringe pump (KD Scientific, Holliston, Mass.). The needle was withdrawn, pressure applied for 1 minute, the tourniquet released, and the incision sutured. Patent blue dye (0.25 mg/ml; ThermoFisher Scientific) was included in the injection fluid to monitor injection success.

### Gene Therapy by Electroporation Delivery

To perform gene therapy, mice were anesthetized with isoflurane. Plasmid DNA was administered to the quadriceps muscles of the mice by intramuscular injection, injecting approximately 50 μg of plasmid DNA in two locations in the muscle group. Four days later, a second dose of DNA was administered in the same way, to increase the dose. Immediately after each DNA injection, electroporation was applied, for example, using a TriGrid electrode and power supply produced by Ichor Medical Systems (San Diego, Calif.).

### Luciferase Live Imaging

Mice to be imaged were anesthetized with 1.5% isofluorane through a nose cone. Luciferin substrate (1 mg/ml PBS; Biosynth, Itasca, Ill.) was injected into the intraperitoneal cavity (100 μl/10 g body weight). Ten minutes after luciferin injection, luminescence was detected using an in vivo imaging system (IVIS Spectrum Pre-Clinical In Vivo Imaging System, PerkinElmer, Waltham, Mass.) and associated software for quantitation. Luminescence images were acquired at exposure times of 1 second or less. Luminescence was quantified within regions of interest in units of photons per second per cm².

### Western Blots

The quadriceps, hamstring, gastrocnemius, and tibialis anterior muscle groups were dissected from mouse hind limbs. Muscle lysates were prepared with RIPA Buffer supplemented with HALT Protease Inhibitor Cocktail (ThermoFisher Scientific) according to the manufacturer's protocol. The supernatant containing protein extract was denatured with Laemmli Sample Buffer (Bio-Rad, Hercules, Calif.) supplemented with 100 mM DTT. In each lane of a 10% TRIS-glycine SDS-PAGE gel (Bio-Rad), 25 μg protein extract was electrophoresed at 70 V in ice cold PAGE running buffer (0.1% SDS, 25 mM Tris, 250 mM glycine). Samples were transferred onto 0.45 μm PVDF membrane (ThermoFisher Scientific) for 60 minutes at 100 mA at 4° C. Membranes were blocked in 0.2% BSA and 2% milk diluted in TBS with 0.2% Tween-20. Dysferlin detection was achieved with a 1:1000 dilution of rabbit-anti-dysferlin (Abcam, Cambridge, U.K.). GAPDH was probed with a 1:10,000 dilution of rabbit-anti-GAPDH (Abcam) in blocking solution. Rabbit antibodies were probed with either a 1:5000 dilution of goat-anti-rabbit IgG HRP secondary antibody (ThermoFisher Scientific) or a dilution of IRDye secondary antibodies (Li-Cor Biosciences, Lincoln, Nebr.). For performing Western blots for CAPN3 expression, a 1:1000 dilution of monoclonal rabbit-anti-FLAG® primary antibodies, a 1:10,000 dilution of monoclonal rabbit-GAPDH primary antibodies, and a 1:1000 dilution of goat-anti-rabbit IgG HRP secondary antibody were used. Blots were developed in Clarify Western ECL Substrate according to manufacturer's protocol (Bio-Rad) and imaged using the ChemiDoc Touch Imaging System (Bio-Rad) or the Odyssey CLx (Li-Cor Biosciences).

### Immunohistochemistry

Muscles were harvested and snap frozen in OCT in liquid nitrogen. Serial 12 μm cryostat sections were obtained throughout the muscles. Sections were fixed in 4% PFA for 10 minutes and washed 3 times with PBS for 1-2 min. Sections were blocked in 10% donkey serum in PBS for 1 hour. Primary antibodies were prepared in block solution and incubated overnight at 4° C. in a humid chamber. The next day, slides were washed 3 times with PBS and stained with secondary antibody for 30 minutes at room temperature. Slides were washed with PBS three times and visualized. Dysferlin was detected with the rabbit NCL-Hamlet antibody (1:500, Leica Biosystems, Buffalo Grove, Ill.), and laminin was detected with a rat alpha-laminin antibody (1:1000, Sigma, St Louis, Mo.). Goat alpha-rabbit conjugated to Alexa 488 (1:1000, Life Technologies, Carlsbad, Calif.) and alpha-rat conjugated to Alexa 594 (1:1000, Life Technologies) were used as secondary antibodies.

### ELISA for Follistatin

Follistatin levels were measured using a follistatin ELISA kit (R&D Systems, London, U. K.) following instructions provided by the manufacturer. A standard curve was constructed using the provided follistatin standards. Protein extract samples from each muscle group were diluted 1:10 in Calibrator Diluent RD5-21. Both standards and samples were pipetted into wells pre-coated with immobilized antibody, followed by incubation with a follistatin-specific enzyme-linked monoclonal antibody. Substrate solution was then added to each well, with color development proportional to amount of follistatin bound. Plates were read with a Tecan Infinite 200Pro microplate reader (Tecan Group Ltd., Mannedorf, Switzerland) at 450 nm with its reference at 570 nm.

### PCR

Presence in muscle of therapeutic plasmid was detected by PCR. Genomic DNA was extracted from muscles using the Wizard Genomic DNA Purification Kit according to manufacturer's instructions (Promega, Madison, Wis.). Plasmid amplicon was generated from 200 ng of genomic DNA (3 min at 98° C.; 20 sec at 98° C., 25 sec at 63° C., 60 sec at 72° C., 30 cycles; 2 min at 72° C.) with the forward primer binding in the firefly luciferase gene (5′-TCTCATCTACCTCCCGGTTTT) and the reverse primer binding in the rabbit beta-globin UTR region (5′-TTTTGGCAGAGGGAAAAAGA) using 2×Q5 MasterMix (New England Biolabs, Ipswich, Mass.).

### Evan's Blue Dye Procedure

To evaluate the membrane permeability of muscle, mice were intraperitoneally injected with Evans Blue dye (EBD; Sigma; 100 μl of 1% EBD in PBS per 10 g body weight). The following day, muscles were flash-frozen and ground with a mortar and pestle. Following an overnight incubation in formamide, EBD was quantified in the supernatant by measuring light emission at 630 nm.

### Statistical Analysis

To determine statistical significance for two groups, comparisons were made using a Student's t-test; for more than 2 groups, the ANOVA test was used. The F test was used to analyze the variance variability of data. Statistical analyses were performed using GraphPad Prism v.7 (GraphPad Software, La Jolla, Calif.). A p-value <0.05 was considered significant.

### Example 1: Plasmids and DNA Vascular Injection

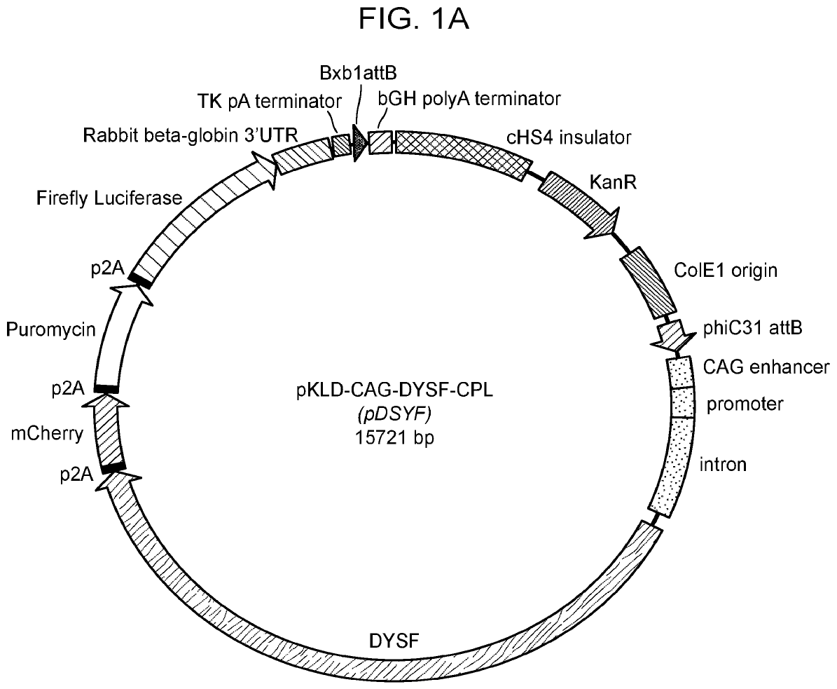

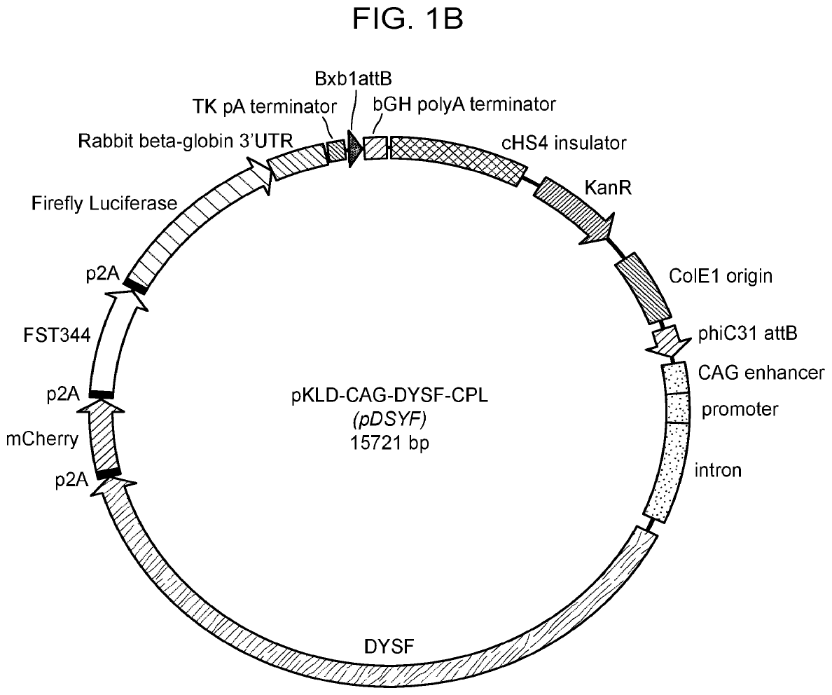

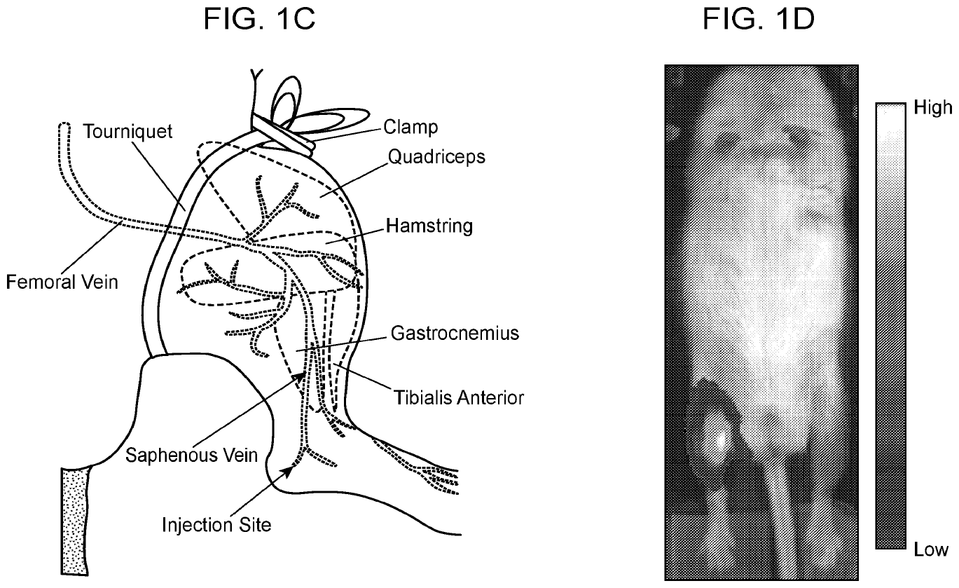

To carry out the plasmid gene therapy approach for LGMD2B, two therapeutic plasmids were utilized (FIGS. 1A & 1B). The plasmids carried the cDNA for human dysferlin (DYSF), a 6.2-kb coding sequence. To obtain strong expression of DYSF, the CAG promoter-enhancer was used, encompassing the widely expressed CMV enhancer, chicken beta-actin promoter, and a hybrid intron. The cHS4 chicken insulator element was also included to combat silencing of gene expression. The rabbit beta-globin 3′ UTR, thymidine kinase polyA sequences, and bovine growth hormone polyA were used for transcription termination. In addition, marker genes encoding mCherry, puromycin resistance, and firefly luciferase, linked by p2A sequences for coordinated expression, were part of the dysferlin expression cassette present in pKLD-CAG-DYSF-CPL, abbreviated pDYSF (FIG. 1A). To obtain a plasmid that co-expressed DYSF and FST, the puromycin resistance gene was removed from the pDYSF, which was not needed in these experiments, and DNA sequences encoding the 344-amino acid isoform of human follistatin were substituted, yielding pKLD-CAG-DYSF-CFL, abbreviated pDYSF-FST (FIG. 1B).

To achieve widespread delivery of therapeutic plasmids to hind limb muscles, a hydrodynamic limb vein injection method was applied. Examples of hydrodynamic limb vein injection methods may be found in, for example, Hagstrom et al. (2004), which utilized naked DNA and distributed it to the major muscle groups of the mouse hind limb. As diagrammed in FIG. 1C, the hind limb circulation was transiently blocked by clamping a rubber tourniquet as high on the leg as possible. Four hundred micrograms of plasmid DNA in Hank's balanced salt solution (HBSS) buffer were injected into the saphenous vein near the ankle using a needle attached to a syringe pump for rapid (8 ml/minute) and controlled delivery of the fluid. Introducing the DNA in a volume of about 1 ml to the leg in the presence of the tourniquet forced the fluid to exit the vasculature, where the plasmid DNA came in contact with muscle fibers, which were competent to take up naked DNA. Successful delivery was monitored by observing transient swelling of the limb, as well as distribution of the blue dye that was included in the injection fluid. To detect expression of the plasmid DNA after injection, expression of the firefly luciferase gene on the therapeutic plasmids was examined by luciferase live imaging. As shown in FIG. 1D, a bright luciferase signal was seen in legs injected with plasmid DNA, whereas no signal was seen in legs receiving buffer alone, nor elsewhere in the body.

FIGS. 1A-1D. Plasmids and DNA injection. (FIG. 1A) Map of plasmid pKLD-CAG-DYSF-CPL (pDYSF) expressing human dysferlin. (FIG. 1B) Map of plasmid pKLD-CAG-DYSF-CFL (pDYSF-FST) expressing human dysferlin and follistatin. (FIG. 1C) Schematic diagram illustrating blood vessels and muscles involved in vascular muscle perfusion method. (FIG. 1D) Luciferase live imaging of BLAJ/NRG mouse legs 4 weeks after injection with pDYSF (left) and HBSS (right), shown with bioluminescence intensity gradient.

### Example 2: Comparison of Single and Double Vascular Injections of pDSYF in Mice in a 4-Week Experiment

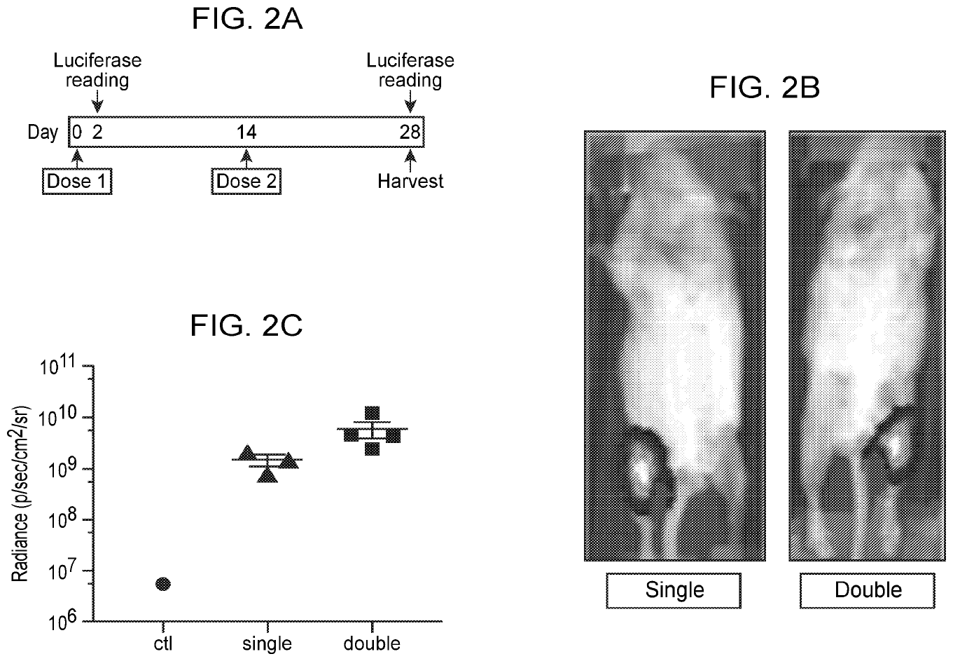

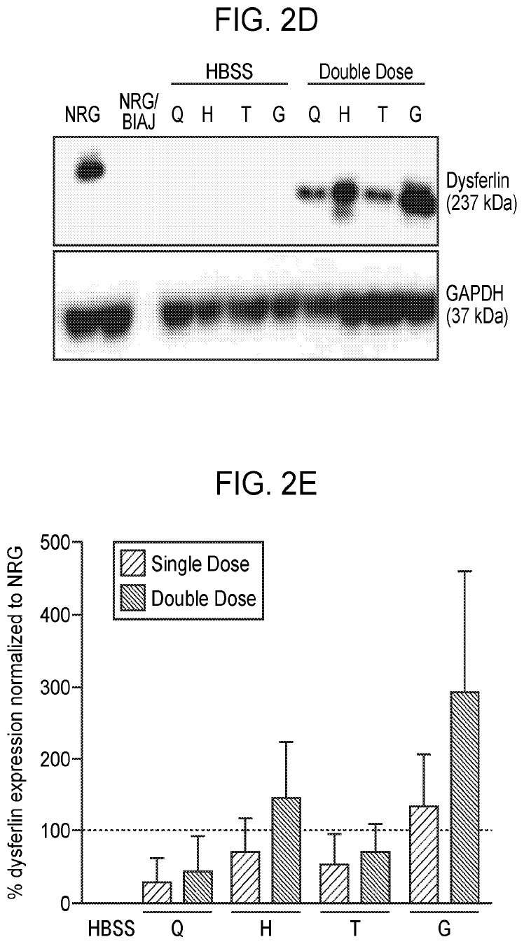

Comparison of single and double vascular injections of pDSYF in B1AJ/NRG mice in a 4-week experiment was performed. The low immunogenicity of naked plasmid DNA itself allowed the potential for multiple doses to be administered. It was previously established in Zhang et al. (2010) that at least six doses of plasmid DNA could be given sequentially, with corresponding increases in the amount of protein delivered and the number of fibers transfected. The effect of two doses of DNA was tested, versus a single dose. As shown in FIG. 2A, mice were given a single dose of pDYSF in one leg, then 2 weeks later, a second dose of pDYSF was given to half of the animals in the same legs. The contralateral legs were injected with HBSS alone. All animals were harvested 4 weeks after the first injection. Luciferase bioluminescence live imaging was done on day 2 to verify injection quality and again just before muscles were harvested. The mice were approximately 6 months old at the time of injection and approximately 7 months old at the end of the experiment. As shown in FIG. 2B, strong luciferase signals were observed in legs that received DNA. FIG. 2C indicated that the average luciferase signal tended to be stronger after 2 doses than after a single dose.

To quantify the levels of dysferlin protein present in the treated muscles, western blots were performed on protein extracted from the quadriceps, hamstring, tibialis anterior, and gastrocnemius muscles, and dysferlin levels were visualized by western immunoblotting with an antibody that recognized the 237-kDa dysferlin protein (FIG. 2D). Equal amounts of protein (25 μg) were loaded in each lane, and the 37-kDa GAPDH protein was imaged as a loading control. Protein extracted from the parental NRG mice, which were wild-type for dysferlin, was used as a positive control representing the normal wild-type level of dysferlin. Protein extracted from untreated B1AJ/NRG mice was used as a negative control and contained no detectable dysferlin, in keeping with the null phenotypic characteristic of the mouse model. Amounts of dysferlin protein present in legs treated with buffer or pDYSF were quantified by normalizing to GAPDH and using the NRG sample to represent 100%. Average amounts of dysferlin present as a percentage of wild-type levels were shown in FIG. 2E for the legs receiving a single or double dose of plasmid DNA. More dysferlin protein was generally observed in the gastrocnemius and hamstring muscles than in the tibialis anterior and quadriceps muscles, and there was a trend toward higher levels of dysferlin protein after two doses. However, because even a single dose yielded values averaging 25 to more than 100% of normal, a single dose of plasmid DNA was administered in further experiments.

FIGS. 2A-2E. (FIG. 2A) Timeline of the experiment; all mice received a first injection of plasmid pDYSF at day 0, while half of the mice also received a second injection of pDYSF at day 14. Luciferase live imaging was performed 2 and 28 days after injections. All mice were harvested at day 28. (FIG. 2B) Representative luciferase live images of mice 28 days after single (left) or double (right) doses of pDYSF. (FIG. 2C) Bioluminescence radiance values were graphed for control (HBSS alone) and single and double doses of pDYSF. While double dose values trended higher, the difference in levels observed between the single and double dose groups was not statistically significant. (FIG. 2D) Western blot images of protein extracted from the four hind limb muscle groups from two representative legs, after injection with either HBSS or a double dose of pDYSF. NRG represents the wild-type level of dysferlin, while NRG/B1AJ represents the null level of dysferlin in B1AJ/NRG mice. (FIG. 2E) Western blot band quantification of all the data showed an upward trend in dysferfin expression in the double dose groups that was not statistically significant. The dotted line represents 100% of wild-type levels of dysferlin. Q, quadriceps; H, hamstring; T, tibialis anterior; G, gastrocnemius. Data are mean±SEM with n=3-4.

### Example 3: Comparison of Single pDYSF and pDYSF-FST Vascular Injections in Mice in a 12-Week Experiment

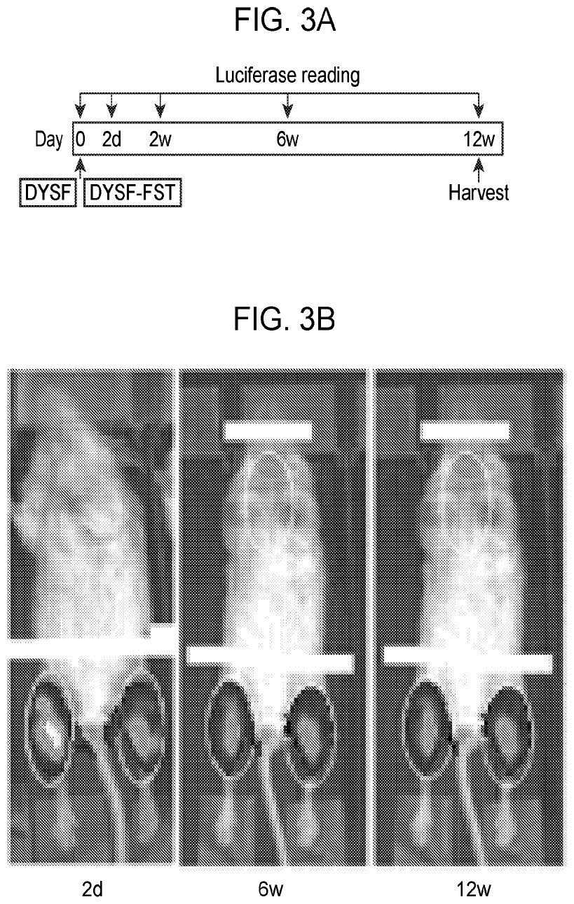

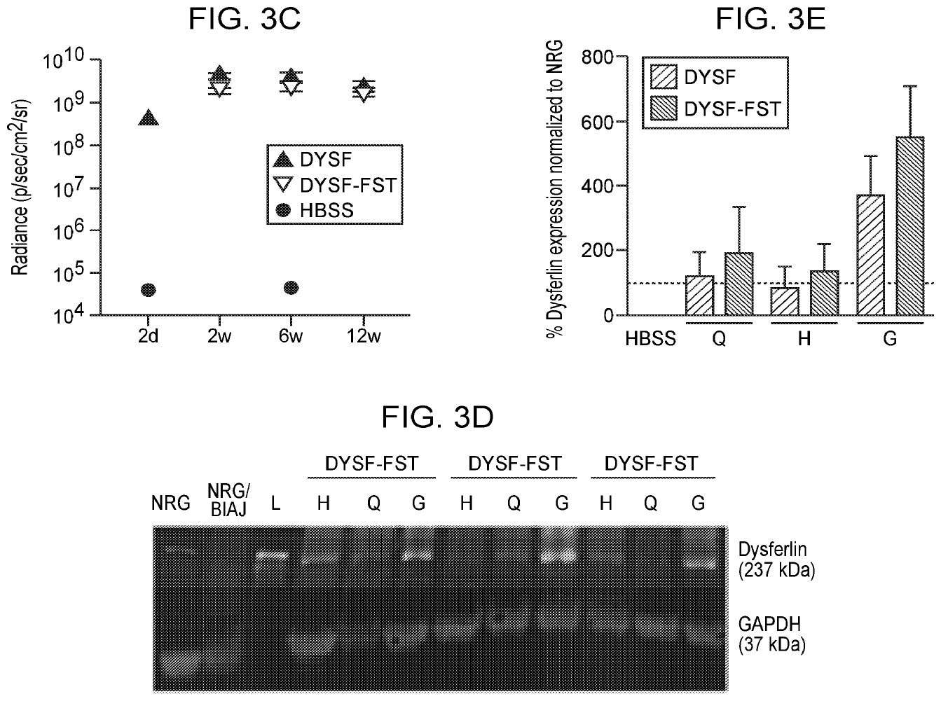

Comparison of single pDYSF and pDYSF-FST vascular injections in B1AJ/NRG mice in a 12-week experiment was performed. To test the durability of gene expression over a longer time period and to measure possible benefit of the treatment, a DYSF gene therapy experiment was carried out that lasted approximately three months. In addition to mice that received the pDYSF plasmid, a group of mice that were treated with a plasmid, pDYSF-FST, carrying both the DYSF gene and the gene encoding human follistatin (FST) was included, to test possible benefit of that combination. As shown in FIG. 3A, the experimental scheme involved a single dose of plasmid DNA or HBSS buffer in each leg, with luciferase live imaging done 2 days after treatment to monitor injection success and at 2, 6, and 12 weeks to examine retention of gene expression. Two mice were treated with HBSS buffer alone, 4 with pDYSF, and 6 with pDYSF-FST. Each group of mice contained both genders, and the mice were aged 8-9 months at the start of the experiment and 11-12 months at the end. Older animals were used to simulate those in which the disease process was well underway.

Mice were treated with pDYSF-FST two days, six weeks, and 12 weeks after DNA injection (FIG. 3B). In FIG. 3C, all the luciferase data, revealing that pDYSF and pDYSF-FST plasmids gave rise to similar levels of luciferase expression were graphed. Luciferase expression rose between 2 days and 2 weeks and then was stable for the 12-week duration of the experiment. The levels of dysferlin protein present in quadriceps, hamstring, and gastrocnemius muscles were evaluated by western blot (FIG. 3D). Dysferlin values were normalized using GAPDH and plotted as a percentage of normal levels of dysferlin in FIG. 3E. Both pDYSF and pDYSF-FST expressed similar levels of dysferlin that averaged about 100% or more of normal levels, with the highest levels in the gastrocnemius muscles. The delivery efficiency for the different muscle groups followed a similar order as what was observed in the previous experiment, although the absolute amounts of dysferlin protein trended higher in the longer experiment (FIG. 2E compared with FIG. 3E).

FIGS. 3A-3D. (FIG. 3A) Timeline of the experiment; all mouse legs received a single vascular injection of either HBSS alone, pDYSF, or pDYSF-FST at day 0, and hind limb muscles were collected at 12 weeks. Luciferase live imaging was performed at 2 days, 6 weeks, and 12 weeks after injection. (FIG. 3B) Representative luciferase images of the same mouse at 2 days, 6 weeks, and 12 weeks after injection with pDYSF-FST. (FIG. 3C) Similar levels of bioluminescence were observed in mice injected with pDSYF and pDYSF-FST. These levels were persistent over the time course of the experiment. (FIG. 3D) Representative western blot images of three mouse legs injected with pDYSF-FST; the H, Q, and G muscle groups were analyzed. Lane L is the protein size ladder. E. Western blot band quantification showed no significant difference in dysferfin expression between the two plasmids FST. The dotted line represents 100% of wild-type levels of dysferlin. Q, quadriceps; H, hamstring; G, gastrocnemius. Data are mean±SEM with n=4-13.

### Example 4: Further Analysis of 12-Week Gene Therapy Experiment in Mice

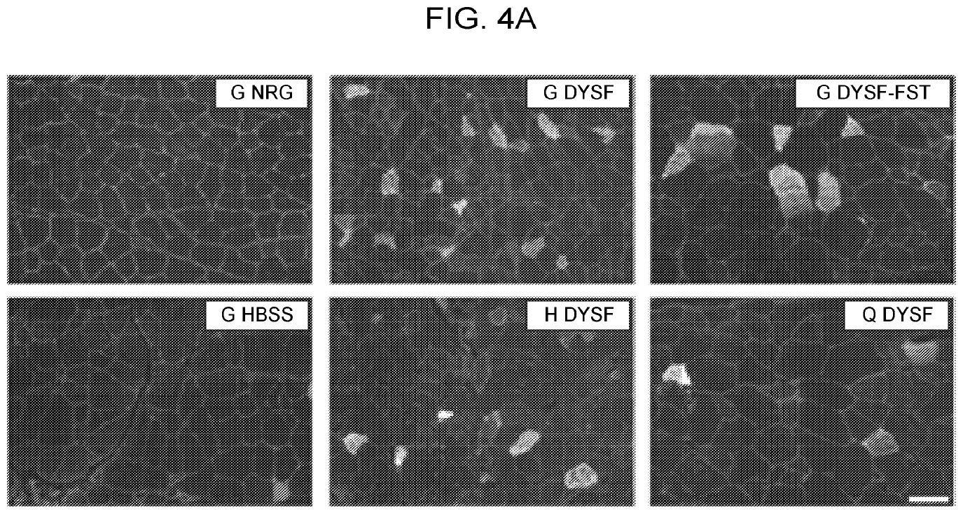

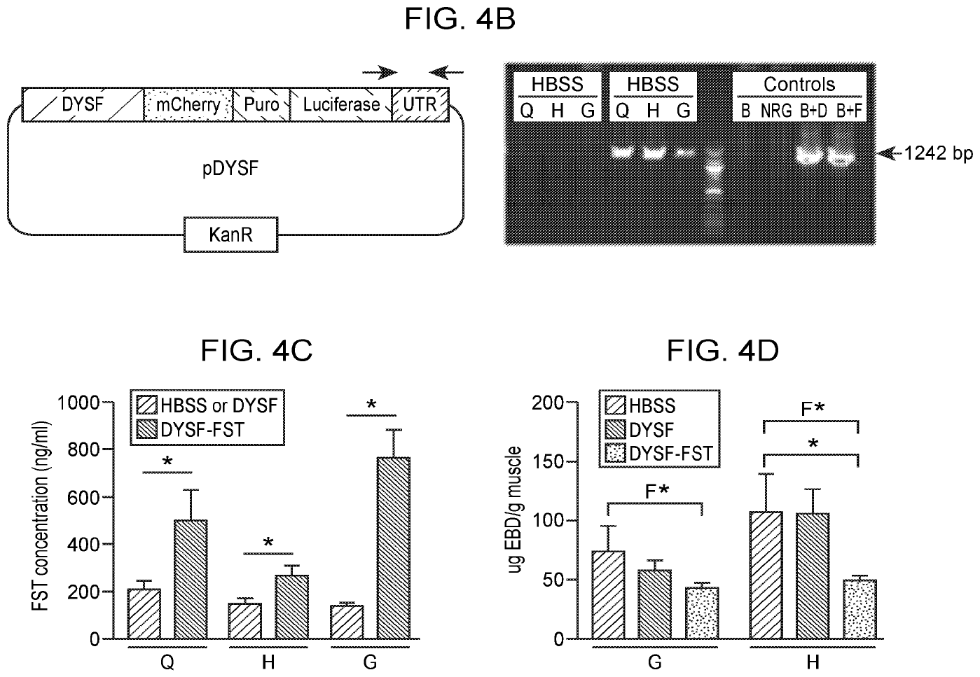

Further analysis of 12-week gene therapy experiment in B1AJ/NRG mice was performed. The muscles in this experiment were analyzed by immunohistochemistry (FIG. 4A). All sections were stained with laminin to demarcate the positions of muscle fibers. In the NRG positive control, dysferlin was visible near the sarcolemmal membrane. In the B1AJ/NRG mouse model of LGMD2B, no dysferlin staining was visible, consistent with the null phenotypic characteristic of these animals and no dysferlin staining was visible in muscles that received only HBSS. Sections from gastrocnemius, hamstring, and quadriceps muscles from legs that received pDYSF showed dysferlin-positive fibers, generally brighter than normal levels of dysferlin with presence of cytoplasmic vesicles, as had been commonly observed also in AAV-mediated gene therapy experiment in mice. A gastrocnemius muscle that received pDYSF-FST was also performed. These sections represented some of the highest densities of dysferlin-positive fibers observed, with fiber counts revealing that 10-12% of fibers were positive for a single dose. Since not all sections had this level of dysferlin-positive fibers, the overall density of positive fibers for this single dose was <10%.

A separate experiment was performed in which about 7-month old mice received one dose of HBSS buffer, pDYSF, or pDYSF-FST. Muscles were harvested after one month to analyze by PCR the presence of plasmid DNA and by ELISA the levels of follistatin protein present in the muscles. PCR was performed on DNA extracted from these muscle samples, using primers that detected luciferase plasmid sequences not present in the genome. As shown in FIG. 4B, a strong PCR band was detected in legs that received plasmid DNA, but not in those that received only HBSS buffer, verifying the existence of plasmid DNA in these muscles. As shown in FIG. 4C, levels of follistatin were significantly higher in muscles that received pDYSF-FST, compared to those receiving pDYSF or HBSS which reflected only the basal level of endogenous follistatin present in mouse muscles.

To analyze whether any improvement in the condition of the treated muscles could be detected, Evan's blue dye (EBD) was injected into the mice intraperitoneally one day before harvest of the muscles in the 12-week experiment. EBD was able to enter muscle fibers only if their sarcolemma was permeable, a condition increasingly present with progression of LGMD2B due to the membrane repair defect present in dysferlin-deficient muscle fibers. The hamstring muscle was the most permeable hind limb muscle to EBD. When the gastrocnemius and hamstring muscles were analyzed for levels of EBD, downward trend was observed in the treated muscles, with a statistically significant drop in EBD uptake present in hamstring muscles treated with pDYSF-FST (FIG. 4D). Furthermore, the animal-to-animal variability as determined by F-test was also lower in muscles that received pDYSF-FST, and this difference was statistically significant (FIG. 4D).

FIGS. 4A-4D. (FIG. 4A) Immunohistochemistry staining on G, H, and Q muscle sections harvested 12 weeks after vascular injection of HBSS alone, pDYSF, or pDYSF-FST. Laminin is stained red, and dysferlin is stained green. A section from the positive control NRG mouse is also shown. Bar=50 μm. (FIG. 4B) Presence of plasmid DNA was detected by PCR in muscles injected with HBSS alone, pDYSF, or pDYSF-FST. Left diagram depicts the position of the primers used to detect an identical 1242-bp region of DNA present on pDYSF and pDYSF-FST. Representative PCR lanes illustrates DNA extracted from Q, H, and G muscles from legs treated with either HBSS alone or pDYSF-FST. Control lanes are B=B1AJ/NRG, NRG, B+D=B1AJ/NRG+pDYSF, and B+F=B1AJ/NRG+pDYSF-FST. (FIG. 4C) Follistatin concentration was quantified by ELISA from muscles injected with HBSS, pDYSF, or pDYSF-FST. Legs injected with pDYSF-FST expressed significantly more follistatin than legs injected with HBSS alone or pDYSF (pooled for statistical purposes) 28 days after the injections. (FIG. 4D) Concentration of Evan's blue dye (EBD) was significantly lower in hamstring muscles of legs injected with pDYSF-FST, compared to legs injected with HBSS alone or pDYSF 12 weeks after injections. Variability was also significantly lower in muscles injected with pDYSF-FST compared to HBSS alone or pDYSF-injected muscles, as determined by F-test. Q, quadriceps; H, hamstring; G, gastrocnemius. Data are mean±SEM with n=4-8 with *p<0.05.

### Example 5: Gene Therapy for Calpainopathy by Delivering Separate Calpain 3 and Follistatin Plasmids

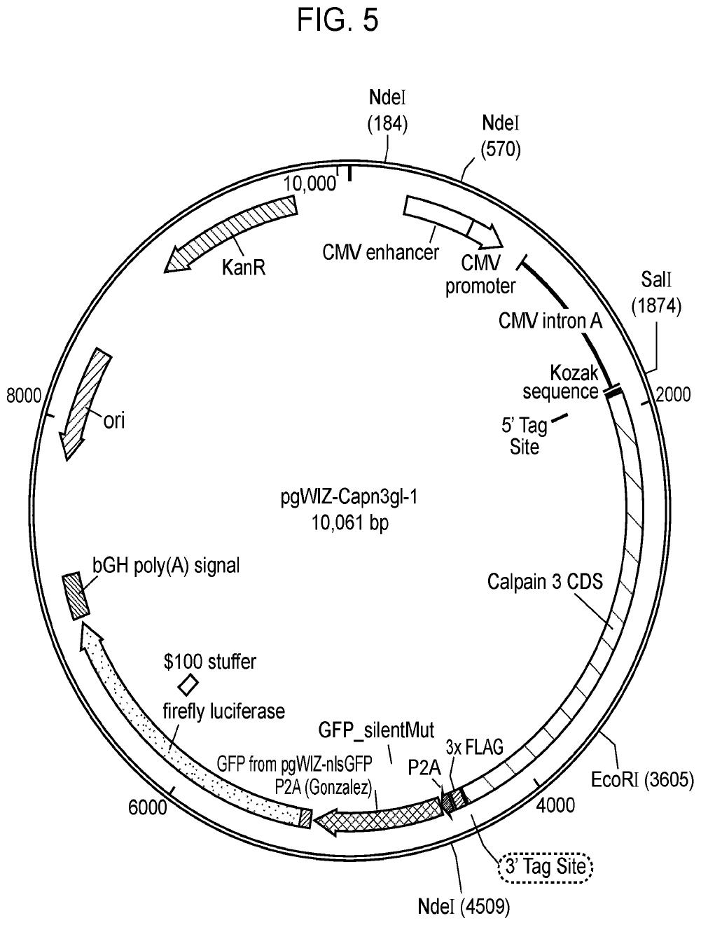

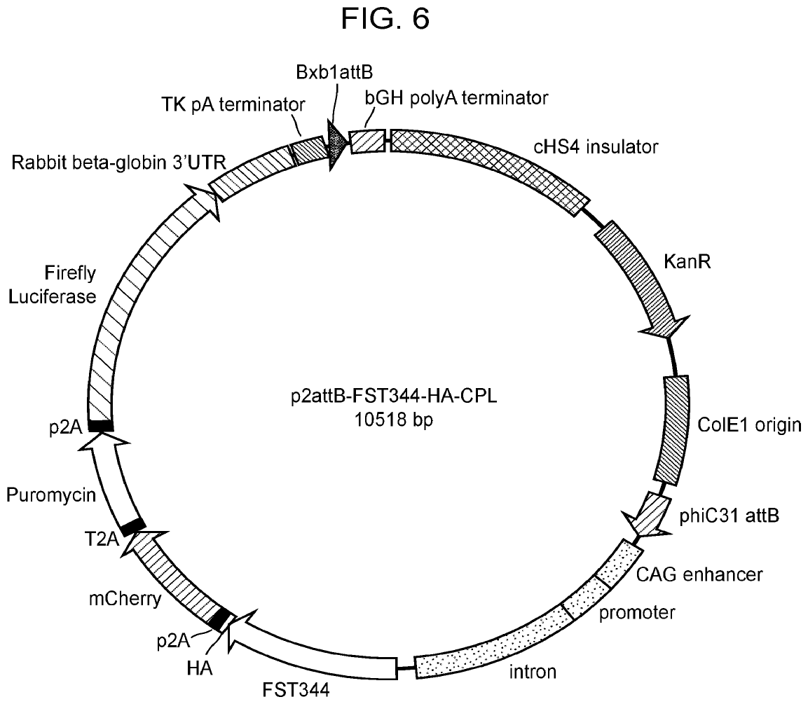

Gene therapy experiments were performed using a mouse model of limb girdle muscular dystrophy 2A in which the CAPN3 gene was knocked out. As shown in FIG. 5, one group of CAPN3-null mice received a plasmid coding for human CAPN3. Another group received a plasmid coding for human follistatin as shown in FIG. 6. A third group received both plasmids, for a synergistic therapeutic effect.

FIG. 5. The plasmid expressed the human CAPN3 coding sequence from an optimized CMV promoter-enhancer-intron. The plasmid co-expressed the GFP and luciferase genes as markers for monitoring gene delivery in muscle. FIG. 6. The plasmid expressed the human follistatin coding sequence from the strong CAG enhancer-promoter-intron, along with the firefly luciferase gene for monitoring gene expression.

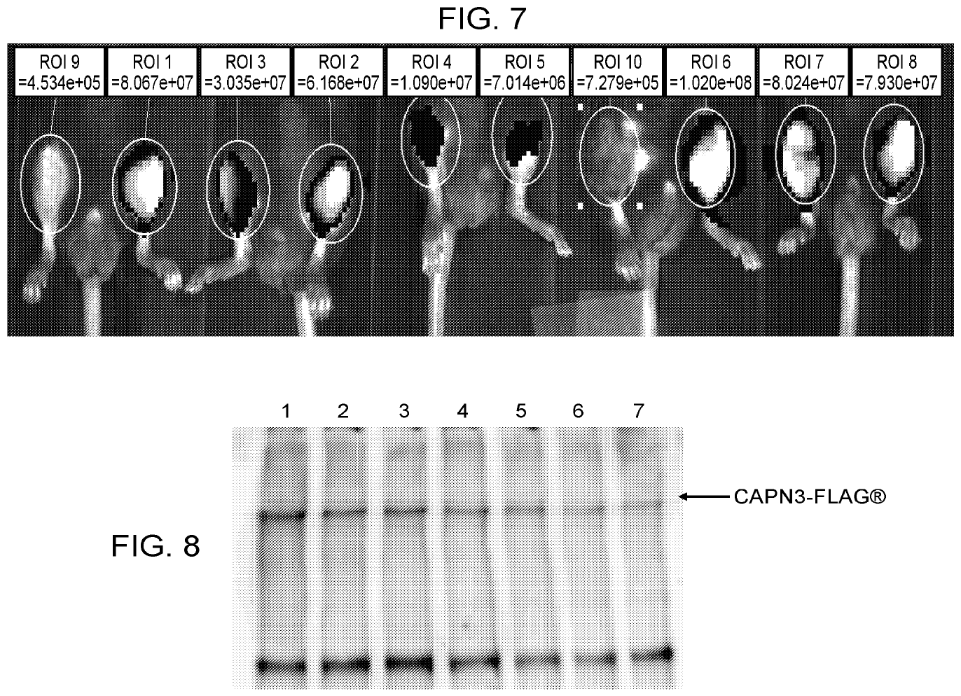

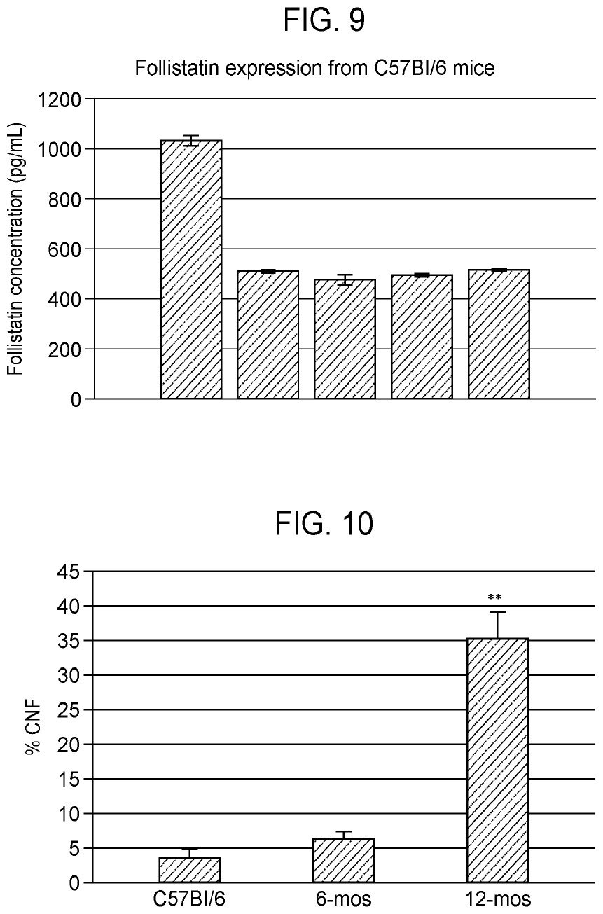

To monitor DNA delivery, mice were analyzed by luciferase live imaging every 2 to 4 weeks as shown in FIG. 7. Approximately three months after gene therapy was initiated, mice were sacrificed and quadriceps muscles were removed. Muscle tissue was analyzed by Western blot to detect CAPN3 expression as shown in FIG. 8, and by ELISA to detect follistatin expression as shown in FIG. 9. Hematoxylin and eosin staining were performed to analyze the frequency of centronucleation in muscle fibers, which provided an indication of the level of muscle damage as shown in FIG. 10. Mice that received the combined therapy of CAPN3 and follistatin plasmids had lower frequencies of centronucleation, compared to untreated CAPN3-null mice, indicating that the gene therapy had a beneficial effect on the treated muscles.

FIG. 7. The luciferase gene was imaged as an indicator of the efficiency of gene delivery of the pgWIZ-Capn3gl expression plasmid for calpain 3, which had been injected and electroporated, in the tibialis anterior muscle of the mice. The results indicated that in both 6-month old (FIG. 7, left three animals) and 12-month old (FIG. 7, right two animals) CAPN3-null mice, the plasmid expressed well, compared to negative controls that received no DNA (FIG. 7, legs 1 and 7 from left).

FIG. 8. A Western blot was performed on muscle samples from CAPN3-null mice previously injected with the pgWIZ-Capn3gl expression plasmid (lanes 2 and 3), as well as other CAPN3 expression plasmids (lanes 4-7; lane 1 is a positive control). Robust production of the correctly-sized CAPN3 gene product was observed, indicating efficient delivery and expression of pgWIZ-Capn3gl in mouse muscle. The lower band was the GAPDH loading control. There was 10 μg protein/well.

FIG. 9. In order to verify production of follistatin from the p2attB-FST344-HA-CPL expression plasmid, an ELISA was performed for human follistatin 1 to 2 weeks after injection of the follistatin expression plasmid in mouse hind limb muscles. The p2attB-FST344-HA-CPL sample, on left expressed significantly more follistatin than the natural background levels in mouse muscle.

FIG. 10. Centronucleation increased in the CAPN3-null mouse over time. Haemotoxylin and eosin staining was used to image sections derived from CAPN3-null mice aged 6- and 12-months. The number of centronucleated fibers versus the number of fibers with peripheral nuclei was calculated. Haemotoxylin and eosin staining showed an increasing amount of centronucleation in CAPN3-null mouse model with age. Gene therapy with the CAPN3 and follistatin expression plasmids reduced the increase in centronucleation, reflecting a healthier muscle after treatment.

### REFERENCES

Mercuri, E. and Muntoni, F. (2013). Muscular dystrophies. Lancet 381, 845-860.
Guiraud S, Aartsma-Rus A, Viera N M, Davies K E, van Ommen G B, Kunkel L M (2015). The pathogenesis and therapy of muscular dystrophies. Ann. Rev. Genom. Hum. Genet. 16, 281-308.
Liu, J., Aoki, M., Illa, I., Wu, C., Fardeau, M., Angelini, C., Serrano, C., Urtizberea, J. A., Hentati, F., Hamida, M. B. et al. (1998). Dysferlin, a novel skeletal muscle gene, is mutated in Miyoshi myopathy and limb girdle muscular dystrophy. Nat. Genet. 20, 31-36.
Bashir, R., Britton, S., Strachan, T., Keers, S., Vafiadaki, E., Lako, M., Richard, I., Marchand, S., Bourg, N., Argov, Z. et al. (1998). A gene related to Caenorhabditis elegans spermatogenesis factor fer-1 is mutated in limb-girdle muscular dystrophy type 2B. Nat. Genet. 20, 37-42.
Bansal, D., Miyake, K., Vogel, S. S., Groh, S., Chen, C. C., Williamson, R., McNeil, P., and Campbell, K. P. (2003). Defective membrane repair in dysferlin-deficient muscular dystrophy. Nature 423, 168-172.
Ho, M., Post, C. M., Donahue, L. R., Lidov, H. G. W., Bronson, R. T., Goolsby, H., Watkins, S. C., Cox, G. A., Brown, R. H. (2004). Disruption of muscle membrane and phenotypic characteristic divergence in two novel mouse models of dysferlin deficiency. Hum. Mol. Genet. 13, 1999-2010.
McGuire, K. K., Lim, L., Speedy, S., and Rando, T. A. (2013). Assessment of disease activity in muscular dystrophies by noninvasive imaging. J. Clin. Invest. 123, 2298-2305.
Lostal, W., Bartoli, M., Bourg, N., Roudant, C., Bentai, A., et al. (2010). Efficient recovery of dysferlin deficiency by dual adeno-associated vector-mediated gene transfer. Hum. Mol. Genet. 19, 1897-1907.
Pryadkina, M., Lostal, W., Bourg, N., Charton, K., Roudaut. C., Hirsch, M. L., and Richard, I. (2015). A comparison of AAV strategies distinguishes overlapping vectors for efficient systemic delivery of the 6.2 kb dysferlin coding sequence. Mol. Ther. —Meth. Clin. Devel. 2, 15009.
Sondergaard, P C, Griffin, D A, Pozsgai, E R, Johnson, R W, Grose, W E et al. (2015) AAV.dysferlin overlap vectors restore function in dysferlinopathy animal models. Annals Clin. Transl. Neurol. 2, 256-270.
Llanga, T., Nagy, N., Conaster, L., Dial, C., Sutton, R. B., and Hirsch, M. L. (2017). Structure-based designed nano-dysferlin that significantly improves dysferlinopathy in Bla/J mice. Mol. Ther. 25, in press.
Jeune, V. L., Joergensen, J. A., Hajjar, R., and Weber, T. (2013). Pre-existing anti-adeno-associated virus antibodies as a challenge in AAV gene therapy. Human Gene Ther. Meth. 24, 59-67.
Bertoni, C., Jarrahian, S., Wheeler, T., Li, Y., Olivares, E. C., and Calos, M. P., and Rando, T. A. (2006). Enhancement of plasmid-mediated gene therapy for muscular dystrophy by directed plasmid integration. Proc. Natl. Acad. Sci. USA 103, 419-424.
Wolff, J. A., Malone, R. W., Williams, P., Chong, W., Ascadi, G., Jani, A., and Felgner, P. L. (1990). Direct gene transfer into mouse muscle in vivo. Science 247, 1465-1468.
Hagstrom, J. E., Hegge, J., Zhang, G. Noble, M., Budker, V., Lewis, D. L., Herweijer, H., and Wolff, J. A. (2004). A facile nonviral method for delivering genes and siRNAs to skeletal muscle of mammalian limbs Mol. Ther. 10, 386-398.
Zhang, G., Wooddell, C. I., Hegge, J. O., Griffin, J. B., Huss, T., Braun, S., and Wolff, J. A. (2010). Functional efficacy of dystrophin expression from plasmids delivered to mdx mice by hydrodynamic limb vein injection. Human Gene Ther. 21, 221-237.
Hegge, J. O., Wooddell, I., Zhang, G., Hagstrom, J. E., Braun, S., Huss, T., Sebestyen, M. G., Emborg, M. E., and Wolff, J. A. (2010). Evaluation of hydrodyamic limb vein injections in nonhuman primates. Human Gene Ther. 21, 829-842.
Wooddell, C. I, Hegge, J. O., Zhang, G., Sebestyen, M. G., Noble, M., Griffin, J. B., Pfannes, L. V., Herweijer, H., Hagstrom, J. E., Braun, S., Huss, T., Wolff, J. A. (2011). Dose response in rodents and nonhuman primates after hydrodynamic limb vein delivery of naked plasmid DNA. Human Gene Ther. 22, 889-903.
Fan, Z., Kocis, K., Valley, R., Howard, J. F., Chopra, M., Chen, Y., An, H., Lin, W., Muenzer, J., and Powers, W. (2012). Safety and feasibility of high-pressure transvenous limb perfusion with 0.9% saline in human muscular dystrophy. Mol. Ther. 20, 456-461.
Fan, Z., Kocis, K., Valley, R., Howard, J. F., Chopra, M., An, H., Lin, W., Muenzer, J., and Powers, W. (2015). High-pressure transvenous perfusion of the upper extremity in human muscular dystrophy: A safety study with 0.9% saline. Human Gene Ther. 26, 614-621.
Lee, S. J. and McPherron, A. C. (2001). Regulation of myostatin activity and muscle growth. Proc. Natl. Acad. Sci. USA 98, 9306-9311.
Rodino-Klapac, L. R., Janssen, P. M. L., Shontz, K. M., Canan, B., Montgomery, C. L., Griffin, D., Heller, K., Schmelzer, L., Handy, C., Clark, K. R., Sahenk, Z., Mendell, J. R., and Kaspar, B. K. (2013). Micro-dystrophin and follistatin co-delivery restores muscle function in aged DMD model. Hum. Molec. Genet. 22, 4929-4937.
Lee, Y. S., Lehar, A., Sebald, S., Liu, M., Swaggart, K. A., Talbot, C. C., Pytel, P., Barton, E. R., McNally, E. M., Lee, S. J. (2015). Muscle hypertrophy induced by myostatin inhibition accelerates degeneration in dysferlinopathy. Hum. Mol. Genet. 24, 5711-5719.
Turan, S., Farruggio, A. P., Srifa, W., Day, J. W., and Calos, M. P. (2016). Precise correction of disease mutations in induced pluripotent stem cells derived from patients with limb girdle muscular dystrophy. Mol. Ther. 24, 685-696.
Miyazaki, J., Takaki, S., Araki, K., Tashiro, F., Tominaga, A., Takatsu, K., and Yamamura, K. (1989). Expression vector system based on the chicken beta-actin promoter directs efficient production of interleukin-5. Gene 79, 269-277.
Niwa, H., Yamamura, K., and Miyazaki, J. (1991). Efficient selection for high-expression transfectants with a novel eukaryotic vector. Gene 108, 193-200.
Chung, J. H., Whiteley, M., and Felsenfeld, G. (1980). A 5′ element of the chicken beta-globin domain serves as an insulator in human erythroid cells and protects against position effect in Drosophila. Cell 74, 505-514.
Zhao, C., Farruggio, A. P., Bjornson, C. R. R., Chavez, C. L., Geisinger, J. M. et al. (2014). Recombinase-mediated reprogramming and dystrophin gene addition in mdx mouse induced pluripotent stem cells. PLOS ONE 9, e96270.
Calos, M. P. (2016). Phage integrases for genome editing. In Genome Editing: The Next Step in Gene Therapy. Eds. Cathomen, T., Hirsch, M., Porteus, M., ASGCT and Springer. Advances Exper. Med. & Biol. 895, 81-91.
Calos, M. P. (2017). Genome editing techniques and their therapeutic applications. Clin. Pharm. & Therapeut. 101, 42-51.
Arnett A L H, Konieczny P, Ramos J N, Hall J, Odom G et al. (2014) Adeno-associated viral vectors do not efficiently target muscle satellite cells. Mol Ther—Meth & Clin Dev 1, 14038.
Lee, S.-J. (2007). Quadrupling muscle mass in mice by targeting TGF-□ signaling pathways. PLoS ONE 2, e789.
Mendell, J. R., Sahenk, Z., Malik, V., Gomez, A. M., Flanigan, K. M., Lowes, L. P., et al. (2015). A phase 1/2a follistatin gene therapy trial for Becker muscular dystrophy. Mol. Ther. 23, 192-201.
Kanimura, K., Zhang, G., and Liu, D. (2010). Image-guided, intravascular hydrodynamic gene delivery to skeletal muscle in pigs. Mol. Ther. 18, 93-100.
Evesson, F. J., Peat, R. A., Lek, A. Brilot, F., Lo, H. P., Dale, R. C., Parton, R. G., North, K. N., Cooper, S. T. (2010). Reduced plasma membrane expression of dysferlin mutants is attributed to accelerated endocytosis via a syntaxin-4-associated pathway. J. Biol. Chem. 285, 28529-28539.
Pearson, T., Shultz, L. D., Miller, D., King, M., Laning, J., et al. (2008). Non-obese diabetic-recombination activating gene-1 (NOD-Rag1 null) interleukin (IL)-2 receptor common gamma chain (IL2r gamma null) null mice: a radioresistant model for human lymphohaematopoietic engraftment. Clin. Exp. Immunol. 154, 270-284.
Wooddell, C. I., Radley-Crabb, H., Griffin, J. B., and Zhang, G. (2011). Myofiber damage evaluation by Evans blue dye injection. Curr. Protocols Mouse Biol. 1, 1-26.
Bertoni, C., Jarrahian, S., Wheeler, T. M., Li, Y., Olivares, E. C., Calos, M. P., and Rando, T. A. (2006). Enhancement of plasmid-mediated gene therapy for muscular dystrophy by directed plasmid integration. PNAS. 103(2): 419-424.
Portlock, J. L., Keravala, A., Bertoni, C., Lee, S., Rando, T. A., and Calos, M. P. (2006). Long-term Increase in mVEGF164 in mouse hindlimb muscle mediated by phage phiC31 integrase after nonviral DNA delivery. Hum Gen Ther. 17:871-876.
Geisinger, J. M., Turan, S., Hernandez, S., Spector, L. P., and Calos, M. P. (2016). In vivo blunt-end cloning through CRISPR/Cas9-facilitated non-homologous end-joining. Nuc. Acids. Res. 1-15.
Calos, M. P. The CRISPR Way to Think about Duchenne's. (2016). N. Engl. J. Med. 374(17): 1684-1686.
Pichavant, C., Bjornson, C. R., Gallagher, T., Neal, T. L., du Bois, H., Bhakta, M., Calos, M. P. (2017). Three novel immune-deficient mouse models of muscular dystrophy. PLOS Currents Muscular Dystrophy. 2017 Sep. 1. Edition 1. doi: 10.1371/currents.md.25e9cd5f4740329f2650972a75230188.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of altering a phenotypic characteristic of muscular dystrophy, comprising:
introducing into the human cell a circular nucleic acid comprising one or more polynucleotide sequences, wherein the one or more polynucleotide sequences comprise at least a follistatin gene sequence and one of a calpain 3 (CAPN3) gene sequence, an alpha-sarcoglycan (SGCA) gene sequence, or a dysferlin (DYSF) gene sequence; and
expressing the one or more polynucleotide sequences in the circular nucleic acid in the human cell, wherein the expressing results in altering the phenotypic characteristic of muscular dystrophy.

2. A method of treating muscular dystrophy, comprising:
introducing into the human cell a circular nucleic acid comprising one or more polynucleotide sequences, wherein the one or more polynucleotide sequences comprise at least a follistatin gene sequence and one of a calpain 3 (CAPN3) gene sequence, an alpha-sarcoglycan (SGCA) gene sequence, or a dysferlin (DYSF) gene sequence; and
expressing the one or more polynucleotide sequences in the circular nucleic acid in the human cell, wherein the expressing results in treating muscular dystrophy.

3. A method of alleviating a symptom of muscular dystrophy, comprising:
introducing into the human cell a circular nucleic acid comprising one or more polynucleotide sequences, wherein the one or more polynucleotide sequences comprise at least a follistatin gene sequence and one of a calpain 3 (CAPN3) gene sequence, an alpha-sarcoglycan (SGCA) gene sequence, or a dysferlin (DYSF) gene sequence; and
expressing the one or more polynucleotide sequences in the circular nucleic acid in the human cell, wherein the expressing results in alleviating the symptom of muscular dystrophy.

4. The method of any one of the preceding claims, wherein the introducing into the human cell provides for integration of the circular nucleic acid into a genome of the human cell.

5. The method of claim 4, wherein the integration of the circular nucleic acid comprises an integrase.

6. The method of claim 5, wherein the integrase is encoded on a nucleic acid expression vector.

7. The method of claim 5, wherein the integrase is a polypeptide.

8. The method of claim 5, wherein the integrase is a phiC31 integrase.

9. The method of claim 4, wherein the integration of the circular nucleic acid comprises a genome-editing enzyme.

10. The method of claim 9, wherein the genome-editing enzyme is encoded on a nucleic acid expression vector.

11. The method of claim 9, wherein the genome-editing enzyme is a polypeptide.

12. The method of claim 9, wherein the genome-editing enzyme is a Cas9 polypeptide, a zinc finger nuclease, a TALEN, or an enzymatically inactive type II CRISPR/Cas polypeptide.

13. The method of claim 4, wherein the wherein the integration of the circular nucleic acid comprises an RNA-guided endonuclease

14. The method of claim 13, wherein the RNA-guided endonuclease is encoded on a nucleic acid expression vector.

15. The method of claim 13, wherein the RNA-guided endonuclease is a polypeptide.

16. The method of any one of the preceding claims, further comprising delivering one or more polynucleotides into a human cell of a target tissue, comprising:
creating an opening in a subject to expose the target tissue;
introducing the circular nucleic acid comprising the one or more polynucleotide sequences into the target tissue; and
applying electroporation to the target tissue.

17. The method of any one of the preceding claims, further comprising delivering one or more polynucleotides into a human cell of a target tissue, comprising:
positioning a device in contact with a subject to restrict blood flow to a limb;
creating an opening in the subject to expose a vessel;
introducing the circular nucleic acid comprising the one or more polynucleotides into the vessel;
applying a pressure;
releasing the device; and
closing the opening.

18. The method of any one of the preceding claims, wherein the circular nucleic acid comprises a promoter and a reporter gene.

19. The method of any one of the preceding claims, wherein the one or more polynucleotides encode a polypeptide.

20. The method of claim 19, wherein the polypeptide is a transcription factor.

21. The method of any one of the preceding claims, wherein the human cell is a muscle cell.
